SEQUENCE LISTING (57 sequences)

SEQ ID NO: 1 - DNA (cDNA), 785 bp; single-stranded, unknown
  immediate source clone: U-U9
cggcacgaggaaaaactangtgagaangagataatcgttgaccgaggnagagaatggcgagcgagaagagcaaaatcctgatcatcgggggcaccgggtacatcggcaagttcatcgtgtttgcgagcgccaggttaggtaaccctaccttcgctctcgtccggagcaccaccgcccccgccggccaacccgagaaggccaagctcctgagcgacttccaggccgccggcgtcaccctcgtccagggggatatntataaccacgagagtctggttaaggcgatcaagctggtggatgtggtcatctcccccgtcggcttcgggcanctgantgatcagaccaagatcatcgacgccatcnaanaagccggaggacacatcaagaggtaccttccatcggagtttggcaacgacgtanaccgaagccatgctgtggagccagcaaagtctacctttgtcgtcaagcaacaaatcanaagggctgttgaggcatcgggtntcccttacacctttgtatcttccaactttcttcggtgggtntttcctcccggtattatgacaggcaggagccactggtcctcccacggacaaggttgtcatcttaggtnacgggaacncaaaacgatcttctcnatgaanacgacttccggacatcccatttantcttggatgatccataacctgaaccaggttctatttcttaaaacttccnccacttcttntctcttancanctcntttccctcttgggaaaaaaatgtcnnctanacttcnaaaagggtttant

SEQ ID NO: 2 - DNA (cDNA), 813 bp; single-stranded, unknown
  immediate source clone: U-U17
cgcgcacgaggaagaaaactaggtgagaangagataatcgttgaccgaggnagagaatggcgagcgagaagagcaaaatcctgatcatcgggggcaccgggtacatcggcaagttcatcgtgtttgcgagcgccaggttaggtaaccctaccttcgctctcgtccggagcaccaccgcccccgccggccaacccgagaaggccaagctcctgagcgacttccaggccgccggcgtcaccctcgtccagggggatatatataaccacgagagtctggttaaggcgatcaagctggtggatgtggtcatctcccccgtcggcttcgggcanctgantgatcagaccaagatcatcgacgccatcaaagaagccggaggacacatcaagaggtaccttccatcggagtttggcaacgacgtanaccgaagccatgctgtggagccagcaaagtcgacctttgtcgtcaagcaacaaatcanaagggctgttgaggcatcgggtatcccttacacctttgtatcttccaacttcttcggtgggtntttcctcccggtattangacaggcaggagccactggtcctcccacggacaaggttgtcatcttangtgacgggaacacaaaagcgatctttctcaatgaanacacatccggacntncacnattaaagcagtggatgatccgaaaacctgaacangttctatatctgaaaccttccgccactcttgtctcatnacaactcatttccctctgggaaaaaaangtcngcaaaactccnaaaaggtctacttcccggaagaaaaatctgaancana

SEQ ID NO: 3 - DNA (cDNA), 746 bp; single-stranded, unknown
  immediate source clone: U-U66
ggcacgaggggaaaaggagatnatcgttgaccgaggagagaatggcgagcgagaagagcaagatcctgatcatcgggggcacggggtacatcggcaagttcatcgtgtttgcgagcgccaggttaggtaaccctaccttcgctctcgtccggagcaccaccgcccccgccggccaacccgagaagggcaagctcctgagcgacttccaggccgccggcgtcaccctggtccagggggatatatataaccacnagagtctggttaaggcgatcaagctggtggatgtggtcatctcccccgtcggcttcgggcagctgagtgatcagactaagatcatcgacgccatcaaagaaagccggaggacacatcaagaggtaccttccatcgggagtttggcaacgacgtanaccgaagccatgctgtggaagccancaaagtcgacctttgtcgtcaagcaacaaattanaagggctgttgaggcatcggggatcccttacacctttgttatcttccaacttcttcggtgggtatntcctccccgtattgggacaggcangaaccactggtcctccccacggacaaggttgtcntcttaggtgaacgggaacaccaangcgatctttctcaatgaaagacaactcgggacatncccnatttaaccantggatgatccnanaacctgaacaaggtctatttctgaaaacttcnccatcttctttttctcataacgaacccntttncctctt

SEQ ID NO: 4 - DNA (cDNA), 795 bp; single-stranded, unknown
  immediate source clone: U-U104
ggcacgaggaaangagataatcgttgaccgaggcagagaatggcgagcgagaagagcaaaatcctgatcatcgggggcaccgggtacatcggcaagttcatcgtgtttgcgagcgccaggttaggtaaccctaccttcgctctcgtccggagcaccaccgcccccgccggccaacccgagaaggccaagctcctgagcgacttccaggccgccggcgtcaccctcgtccagggggatatatataaccacgagagtctggttaaggcgatcaagctggtggatgtggtcatctcccccgtcggcttcgggcagctgagtgatcagaccaagatcatcgacgccatcaaagaagccggaggacacatcaagaggtaccttccatcgggagtttggcaacgacgttanaccgaagccatgctgtggagccagcaaagtcnacctttgtcgtcaagcaacaaatcanaagggctgttgangcatcgggtntcccttacacctttgtatcttccaacttcttcggtgggtatttcctcccggtattangacaggcaggaccactggtcctccccacngacaaggttgtcntcttaggtgacnggaacacaaaancatctttctcnatgaagacaactccggacatacncnattaaagcngtggatgatcccaaaacctgaacaagttcctatntcttgaaaactttcccgcccaaccatcctttngttctcccnttaaaccnaacctccattttcccctccttggggaaaaaaaaaaggtccggcnaanaacttccnaaa

SEQ ID NO: 5 - DNA (cDNA), 797 bp; single-stranded, unknown
  immediate source clone: U-U13
cggcacgaggnagaaccttttgacagagttgttgtcatggcaacaaaagcttctctctccataaaaggctttgccttgctggtttcagtccttgtagcagttccaacaagagtgcaatcgattggtgtctgctacggcatgctcggcaacaatcttcccccgcccagcgaggtggtcagtctctacaaatccaacaacatcgcgaggatgagactctacgatccaaaccaagccgccctgcaagccctcaggaactccaacatccaagtcctgttggatgtcccccgatccgacgtgcagtcactggcctccaatccttcggccgccggcgactggatccggaggaacgtcgtcgcctactggcccagcgtctcctttcgatacatagctgtcggaaacgagctgatccccggatcggatctggcgcagtacatcctccccgccatgcgcaacatctacaatgctttgtcctcggctggcctgcaaaaccagatcaaggtctcgaccgcggtcgacacgggcgtcctcggcacgtcctaccctccctccgccggcgccttctcctccgccgcccaggcgttacctganccccatcgtgcagttcttggcgantaacggaacgccgctcctggtcaatgtgtaccttattttaactacaccggcaacccggganagatctcgctgcctacnccctgttcacggccncggggtctcntgcaggatgggcgaattccnctatcanaanctgttcantccatcttcnaaaccggtcttcccgggctggaaaaaattggaag

SEQ ID NO: 6 - DNA (cDNA), 792 bp; single-stranded, unknown
  immediate source clone: U-U136
ggcacgaggagaacccttttgacagagttgttgtcatggcaacaaaagcttctctctccataaaaggctttgccttgctggtttcagtccttgtagcagttccaacaagagtgcaatcgattggtgtctgctacggaatgctcggcaacaatcttcccccgcccagcgaggtggtcagtctctacaaatccaacaacatcgcgaggatgagactctacgatccaaaccaggccgccctgcaagccctcaggaactccaacatccaagtcctgttggatgtcccccgatccgacgtgcagtcactggcctccaatccttcggccgccggcgactggatccggagggaacgtcgtcgcctactggcccagcgtctcctttcgatacatagctgtcggaaacganctgatccccggatcggatctggcgcagtacatcctccccgccatgcgcaacatctacaatgctttgtcctcggctggcctgcaaaaccagatcaaggtctccgaccgcggtcgacacgggcgtcctcggcacgtcctaccctccctcccgccggngccttctcctccgccgcccaagcgtnctganccccatcgtgcanttctttggcgaaataacggancgccgctcccnggtccatgtttttcccttattttaactacnccggcaacccnggaaagatttcgctgcccttacgccccctggtttccnnggggcttcccgggcgtttcttcctttgccagggantnggggcgaattcccnncttttccanaaaccttgttccaacnccc

SEQ ID NO: 7 - DNA (cDNA), 855 bp; single-stranded, unknown
  immediate source clone: U-U21
cggcacgagtctctctctgtctctccgtcgtctcgttgtctgttcgttagggcttgcgatcgccaccggtcgcgagggttggagccatggaattcttgcggtttggggctgaggtggcggcaggagaggaagccgcgacggggtactggatgcgatggcagacgctcgtctgcgctctgatcgtcgtcgccccggcggtcgccgcggtggttgtcgcggcgcgggcgccggcgccggcgcgaccgctcagggccgtcgatctctgggcgccgtgctgggccgggatgcacccggcctggcttctcgcgtaccgcggcttcntgttcttggccatggcgtggctactcttccanatgattctgtttcgcggattctccgcgttctacttctatactcagtggacgtttgccttagtcattgtctattttgcgatcgcaaccattatatccgcccatggctgctggctttactcaaagagaagtattatgccagatcaggaggtcaacagattcctaaatggtggttttgagcanaatagtcctatgactctacctttgaggaccaacaaaaatatgaatgttataagattgcaaagctatcntgaacaggangctgatgaanaaaaanctggattttggggtcctgctatgcnactngtctatcaaaaccattgcangtgctgtaattgttgaaanacattgtattttgggntcntnctaataccatatttgtcttctataaatttcaggctaaatgctataatgggctgcatgcattctcttaatgctgtgtttcttcnaatccaacacttttctcaataacctgccattcccntntttcnaatggcatttttgc

SEQ ID NO: 8 - DNA (cDNA), 722 bp; single-stranded, unknown
  immediate source clone: U-U31
cggcacgagagagagagagagagagagagagagagagagagagagagagagagagagagagagagagagcctagaccggcatcntcgtgcttgccgaggggcggcttatgantcttggttgtgccactgggcatcccagcttcgtcntgtcctgctccttcaccanccangtgatngcacnactgganttatgganggaaanggcgaccggcnngtacganaagaangtctatgtgctgcccangcntctggatgagagggtggcngcgctccncctcggcaagctgggtgccncgctcaccnagcttacnccntcgcgggctgattacattagcgtcccgatcgagggaccctacangcctgctcactacnggtattngggttgcttntgcnagagacgatgatnatanntcggagcactggcnttttcgactannagtttgaccgatggctatgttcgttttgcttttcaccttttgtcttcccatctttgctggttcacctatggacgtttgttccatttggatgtnntgagaaatgctgatggcattttcggaaanaaaaanatntaaaatcncgagagttcttctanagcggccgcgggcccntcnnttttcccccgggtggggtaccangtttgttgtncccnttccccctntttgagtccttttactncccctggccgtcgttttatacntcgttgactgggaanaccctgccnt

SEQ ID NO: 9 - DNA (cDNA), 793 bp; single-stranded, unknown
  immediate source clone: U-U131
ggcacgagccttgcccaactccttcgattcgttccaacgatcggagaatggcgctgctggtggagaanacgtcgacgggacgcgagtacaaggtgaaggacctttctcaggctgacttcggccgcctcgagatcgagctggcggaggtggagatgccgggcctcatggcgtgccgcgccgagttcgggcccgccaaacctttcgccggcgcccgcatctccggctccctccacatgaccatccagaccgccgtcctcatcgagaccctcaccgccctcggtgctgaagtccggtggtgctcctgcaacatcttctccacccaggaccacgccgctgccgccattgcccgcgactccgccgccgtcttcgcctggaagggagagaccctcgccgagtactggtggtgcaccgagcgatgcctcgactggggccccaacggtggccccgacctcatcgtcgacaaagggggtgacgccactctcctcatccacgaaggcgtcaaggctgangaagantacgaaaaagaccggcaagctgcccgatccggcctccacggaaaacgccgaattccaaatcgtgctggggattatccgcnancggctccaggtcnaaccccnaaaattaccgcccagatnaaagaaccctcctccgctttttgaagaaaacancnctggcgttcancgaatctaccanatgccagggccancnggggccccttgcttctttccccccncccantcnaacnttccnacgaattcccgttttacccnaaaaaccaanttttgaacaacct

SEQ ID NO: 10 - DNA (cDNA), 847 bp; single-stranded, unknown
  immediate source clone: U-U32
cggcacgaggcgacgccggccttcgcctccgcgtctccctccacctccactgccaccgccgccctcgcctccccctccccaaatcggtcgactccgccgccgctacagaccccgacatcctattcactgaccgcgccggccgccgccgtgccgactgcctctccttcgctgtcgacgacctccctgtcctcgacggcaggaccccgatggaagcctacgaggagttcttccggagcttccgcctcgccttcgctgacttctttggttccgtcattacggatatcacgatcggtctcgggccaaatggcgagctccgttacccttcgtttcctcccactggaagcaatcgtttcaccggtgtaggagaattccagtgttatgacaaatacatgcttgccgatctcaaacgacacgcggaggaaaccggcagcccattatggggcctctctggtcctcacgatgcccctgggtacaaccagtctccggacttcggcaactttttcaaggaccatggcggctcctggganacaccctacgggcaattcttcctctcctggtacaccggcaagctcctgtctcacggtgacggcctgctctcggtcgcgtcagaagtattggtgatttgcctgtcgcgctctcgggcaaggttccactttctgcatttgttggcaacgaaactcggtcgcgcccgtcttancttgaaggccgggttctntnacacttgacggnanaanaacnggtttcaaggatgttggctaanatctcccgaaancntcctgcancatgattcatcccgggcntggacttctcngaaggggaacantcctcagggtgttccggtctgccc

SEQ ID NO: 11 - DNA (cDNA), 822 bp; single-stranded, unknown
  immediate source clone: U-U55
ggcacgaggcatggagaaccctgttgcgatgggcatcatggaggagcttgaggcccagagcgaggtgtggaaccacatttttagattcatcacctccatgtccgtcaagtgcgcggtggagctcagggtccccgacgccatccacgcccacggcggcaacgcaactctccctcagctggccgccgccctcagcctccctcccgccaagctcgccgacctccgacgcctcatgcgcatgctggtccacgccggctgcttcgccaagcaggaagacnacgtgtacgccctcacgccatggtcgaggctcctgntgagctccgagcacaccgcggtagccccgttcgtggtgtggatgctccacccgctcatggtgcagtcgtggcactcgctgggggcgtggttccacgggagggcgcccacccccttcgccgcaacccacgggaaaggggatcttcgagacnacacncnaacagccggggttcgcggccgttcttcancnangcnatggcgaacgactgtcggctggtgggaaaggtgttggtaaanaacatncggatgtgttgganggancncnggtccatngtggaagttggtggcgcaccggcacccttttnggccattgttggccgaaggctttcccgccaacatnaaatngcaccgttctccnantttgccccacttttctggccncngccgccggggcccccncnnaccnaaacaacttggatgtttttngggggaanacatnttccaacattttacanccgcncgacnttttttacncaattggttccnggcncnaatggaaatantnctgnattt

SEQ ID NO: 12 - DNA (cDNA), 743 bp; single-stranded, unknown
  immediate source clone: U-U72
ggcacgaagagccctgtaccgganagcatggagaaccctgttgcggtgggcatcatggaggagcttgaggcccagagcgaggtgtggaaccacatttttagattcatcacctccatgtccgtcaagtgcgcggtggagctcagggtccccgacgccatccacgcccacggcggcaacgcaactctccctcagctggccgccgccgtcagcctccctcccgccaagctcgccgacctccgacgcctcatgcgcatgctggtccacgccggctgcttcaccaagcaggaanacnacgtgtncgccctcacgccatggtcgangctcctggtgagctccgancacacctcggtggccccgttcgtggtgtggatgctccacccgctcatggtccagtcgtggcactcnctgggggcgtggttccacgggaagggcgcccacncccttcnccncnacccncgggaaggggatcttccanacgacacncgaacatccggggttcgcgggcgtcttcaactangcgatggcgaacgactgtcngctggtgggaaangtnttggtaaaaaaaccttccggaagtgttnganggaaccccgtccaatngtggaanttggtggcngccggcacccttncgggcnatcgttgtcgaagnccttcccncacnttanattncaccgtcctccaattntccccantttctttngccgcnggnnncgggccccnggtnanccnaaancntttntttt

SEQ ID NO: 13 - DNA (cDNA), 806 bp; single-stranded, unknown
  immediate source clone: U-U68
tgttctctccttcgatcacatctttttgctctgggaaacgtgagaggtgagacgagggcggcaatgacggcgggtttaagatggattcctcctctgcttcttcttcttctgggcttcctgctggttttgaacggaggtcgggggtggattggaagcgagaggtcctctggctcgaggaatggcggagcatcgcggaggagcttgagagaggcctccgcgaacgcgaccagcgccgatgcttccttggaagagagggctgtaacccgggcagcagaagccgcagtcgacgaccccgaggaggttgcttcgacggtcctgatgaccataatcaacagcacggctcgcagatctcttggttatctgtcgtgcggttcaggcaacccgatcgacgactgctggcggtgcgaccctgattggcatgtcaacagaaaaaagctcgctgactgcggcattggctttggacgcaacgcnatangtggccgcgacggggaatttgttcgttgtgacagactccggggacnatgatccccgtgaatcctcgcccgggaacacttanataccccgtcntccaangaagttgccccctctgggatcccctttaaacncnaantggaaatctcnctcnanggaagaactcnttatgaacagctttaanacnatcgatggacncngtgtcctcgtccacattgccaatggcgcctgcntcaccanccaatttcntccccaacntcttcttccntngcctccnctccccaatgcaacccnccgggaattcctngtccccnctccccttctcctatggatng

SEQ ID NO: 14 - DNA (cDNA), 687 bp; single-stranded, unknown
  immediate source clone: U-U69
ggcacgagggagaacaaaaatggcagcattcttgttcttcctcaccatcgcagccttcactgcccccatctactcttctcgtgcacccttaacgtcggcagctgtccgcgaccctgaattagtagtacaggaagtacaaagaagcttgaacgtgtcgcggcggcgactgggctacttgtcatgcggcaccggcaatccgatcgacgactgctggcggtgcgaccctgactgggctgacaaccggcagcggctcgcggactgcgccatcgggttcgggaagaacgcgattgggggcagggacggcgagatatacgtggtgaccgacagtggcgacnacnaccccgtcaatccgaagccgggcacgctccggtacgccgtcatccangagggagccgctgtggatcatcttcaagcgcgacatggtcatccagctgaaggaaggagctcatcatgaaactcccacanagaccatcgacggccggggcgccagcgtccacatctccggggggccgtgcatcaccaccagtncgtcnccaacatcatcatccacgggcgtccacttccacnaatgcaancagggcggggaacgccttacgttcgcgactcccccagggcactacgggtggcgcaccggtgtccgaacggcnanggggttttcatcttccgcgggaaccacttctggggtca

SEQ ID NO: 15 - DNA (cDNA), 741 bp; single-stranded, unknown
  immediate source clone: U-U84
tccacattgccaatggcgcctgcatcaccatncagtacatcaccaacgtcatcatccatggcctccacatccacgactgcaagcccaccgggaatgccatggtccgcagctctccttctgactatggatggagaaccatggctgatggggatgccgtttccattttcggctccagccacatttgggtggaccactgctctctgtccaactgcgccgatggacttgtcgatgccgtcatgggctccactgccattacggtctccaacaattacttcacccaccacaatgaggtcatgcttttgggacacactgattcttatgcaagggacagcatcatgcaagtaacgatcgcatttaaccattttggtgaaggtctgattcagagaatgcccaggtgcaggcatggctacttccacgtggtaaacaatgactacacgcactgggagatgtacgccattggcggtagcgcgaatccaacgatcaacagtcaaggcaaccgataccttgcgccgaccaatccatttgcaaaggaagtaacaaaaagggtggacacagatcaaagcacgtggaaaaactggaattggangtcngaaggtgacctgcttctgaatggtgcttttttcacccttccggtgcangggctttcacccanctacgcacgggcctccactttgggggcaaccctcttccttngttganacactgactctgatgctggggtcctgttttgccaattcggnc

SEQ ID NO: 16 - DNA (cDNA), 727 bp; single-stranded, unknown
  immediate source clone: U-U89
ggcacgagttctctccttcgatcacatctttttgctctgggaaacgtgagaggtgagaagagggcggcaatgacggcgggtttaagatggattcctcctctgcttcttcttcttctgggcttcctgctggttttgaacggaagtcgggggtggattggaagcgagcggtcctctggctcgaggaatggcggagcatcgcggaggagcttgagagaggcctccgcgaacgcgaccagcgccgatgcttccttggaagagagggctgtaaccagggcagcagaagccgcantcgacgaccccgaggaggttgcttcgacggtcctgacgaccataatcaacagcacggctcgcagatctcttggttatctgtcgtgcggttcaggcaacccgatcgacgactgctggcggtgcgaccccgattggcatgtcaacagaaaaaagctcgctgactgcggcattggctttggcccgcaacgcgatangtggccgccacnggganttgtncnttgttgacagactccggggacgatgatcccgtgaatcctcccccggaacacttaatattccccgtcatcccaganctncccctctggatcnccttttaaacacgacattggaaatcncccccnggaagaactcctttntaaccgctttnnaacatcnatggacncgtgtccacnttccacntttgccaatggcgcctgcctcnccanccnatttaatcncc

SEQ ID NO: 17 - DNA (cDNA), 842 bp; single-stranded, unknown
  immediate source clone: U-U115
ggcacgagggagaacaaaaatggcagcattcatgttcttcctcaccatcgcagccttcactgcccccgtctactcttctcgtgcacccttaacgtcagcagctgtccgcgaccctgaattagtagtacaggaagtacaaagaagcttgaacgtgtcgcggcggcgactgggctacttgtcatgcggcaccggcaatccgatcgacgactgctggcggtgcgaccctgactgggctgacaaccggcagcggctcgctgactgcgccatcgggttcgggaagaacgcgatttggggcagggacggcgagatatacgtggtgaccgacagtggcgacnacgaccccgtcaatccgaaaacgggcacgctccggtacgccgtcatccaggaggagccgctgtggatcatcttcaagcgcgacatggtcatccagctgaaggaggagctcatcatgaactcccacaagaccatcgacggccggggcgccagcgtccacatctccggcgggccgtgcatcaccaccagtacgtcaccaacatcatcatccacggcgtccacatccacgaatgcaagcagggcgggaacgcgtncgttcgcnactccccaagggcactacgggttggcgcacngtgttcngacggcgacgggtgtccatcttccgccggaaccactctgggtccaaccactgcancgcttgttccaacttggcccaccnaantgggccttccntcccaacccccatttccttgggattnccccccgccaattcccccattttccaacnaacttnctttgaacccncccntgaanaaggtccttgctgctgggtcccaccaacaatttna

SEQ ID NO: 18 - DNA (cDNA), 724 bp; single-stranded, unknown
  immediate source clone: U-U117
ggcacgagtctccttcgatcacatctttttgctctgggaaacgtgagaggtgagacgagggcggcaatgacggcgggtttaagatggattcctcctctgcttcttcttcttatgggcttcctgctggttttgaacggaggtcgggggtggattggaagcgagaggtcctctggctcgaggaatggcggagcatcgcggaggagcttgaganaggcctccgcgaacgcgaccagcgccgatgcttccttggaagacagggctgtaaccagggcagcagaagccgcagtcgatgaccccgaggaggttgcttcgacggtcctgacgaccataatcaacagcacggctcgaanatctcttggttatctgtcgtgcggttcaggcaacccgatcgacnactgctggcggtgcgaccccgattggcatntcaacagaaaaaagctcgctgactgtggcattggctttggccgcaacccgatangtggccgcgacgggganttgtncgttgtgacagactccggggacgatgatccccntgaatcctcccccgggaacacttanatncccgtcntccnngaantttcccctctggatcncctttaaacccacntgganatcccccncnaggaagaactccttatgaacngctttttaaaacatcgatnggacccngtttccacttccncattngccnaattgggcgccctggccntccncccntccccaa

SEQ ID NO: 19 - DNA (cDNA), 797 bp; single-stranded, unknown
  immediate source clone: U-U80
ggcacgagggacaagcgatggcagcagaattgtctcccacgctgagcaagagcatctttgaaggagccggtggatcttactctacttggtcaggtgccgatctcccccttcttactgatgcaaagctcggcggaggcaagcttgtcctgaaaccactgggcttggcgttgcctcactattccgactcatcgaaagtcggctatgttcttgaaggaagagcggtggtggggctaacactctatggagagaccgagcagaggatactgctgcttgagaagggagatgtggtagcggtggtcatggggagcctcacgtggtggtacaacgaggaggaggactccgacttctccatcgccttcttaggcgataccgcgacagctgtgcgaccgggcgacatcgcctacttcttcttggcaggatccctaggaagtgctccatggcttttcgacggaaattcctcancagggcctgcggtttaaggggatgcggaagctgangagctcttcggaagccaacctggtactctaatcatcacacngcagcaaaagctgcctggcctcagancatcccgagctgacagcnaagggatatcntaaacgctgancgcgtancggcatatatcaatgtgaaaagtggtggctgttctgcctcggtgacccttnatnaactggcngcnctgggaagatcaggttctccgtcnacctccaaaaatccancctaaccccgtgccttgccggggttcttcnttgaatcnnctgttcanctgaaatttttccccaaggccntggaacaattct

SEQ ID NO: 20 - DNA (cDNA), 742 bp; single-stranded, unknown
  immediate source clone: U-U90
ggcacgagatgactctctcggcggtagcatcggatgccgatgattcggtcgcttatacattcgcttcgcgatacgttcgcgaggctcttccccggttcaggataccggagcagtcgatccccaaggatgcggcgtaccagatcatcaacgacgagctgatgctcgacgggaacccgcggttgaatctggcgtcgttcgtgacgacgtggatggagccggagtgcgatcgcctcatcatggcggccgtcaacaagaactacgtcgacatggacgagtaccccgtcaccaccgagctccagaatcgctgcgtaaatatgatagcccaccttttcaatgccccaattggggaanacgaaacggctgttggagttggaactgtgggttcctccagaagcaatcatgcttgcaggacttgcattcaagaggaaatggcagaacaaaagaaaggcagagganaagccttacgacaaacccaacnttgttaccggtgcaaatgttcaggtttgctggganaaatttgcaaggtattttgaagtttgaactgaaagaaattgaagttgaaagaaggatattatgttaatggatcctgccaaggcagtanaaatggttgatgaaaaatacatatgtttttgctgccatctttgggttcaactctccctgggaaaatttgaanatgttaaccttctaaatgatctcctgacngaaaaaaacccagaaactgggttggaccccccntacttgt

SEQ ID NO: 21 - DNA (cDNA), 807 bp; single-stranded, unknown
  immediate source clone: U-U92
ggcacgagctctagccgatgactctctcggcggtagcatctgatgccgatgattcggtcgcttatacattcgcttcgcgatacgttcgcgaggctcttccccggttcaggataccggagcagtcgatccccaaggatgcggcgtaccanatcatcaacgacgagctgatgctcgacgggaacccgcggttgaatctggcgtcgttcgtgacgacgtggatggagccggagtgcgatcgcctcatcatggcggccgtcaacaagaactacgtcgacatggacgagtaccccgtcaccaccgagctccagaatcgctgcgtaaatatgatagcccaccttttcaatgccccgattggggaanacnaaacggctgttggagttggaactgtggggtcctcaaaagcaatcatgcttgcagggcttgcattcaagaggaaatggcanaacaaaagaaaggcagagganaagccttacgacaaacccaacattgttaccggtgcaaatgttcnggttttgctgggaanaaatttgcaaggtattttgaaatttgaactganagaantgaanttgaaagaaggatattatgtttatggatcccngcccaaggcagtanaaatggttgatgaaaataccaatttntgttgcttgcccatctttgggttccactctcnctggganaattttgaaaaatgttnagcttctaaatgatctccnggaccnaaaaaanncccnnaanctnggttgggacccccccntaccttttcnatncctgccanttnggggattcnntancgcctttcctnctatc

SEQ ID NO: 22 - DNA (cDNA), 770 bp; single-stranded, unknown
  immediate source clone: U-U91
ggcacgagagaaaaaattgtaaagtttgactttcatggacagcctgcggagctcaagcatggtagccttgtcatagcagcaattacaagctgcacaaacacatcaaatcccaatgttatgcttggtgctggtcttgttgcaaagaaggcctgtgaattaggtctacaggttaagccttggatcaaaaccagccttgctccaggttctggagttgttaccaagtatttgcttaagagtggcctacaagaatatttgaatcagcaaggatttaatattgttgggtatggctgcacaacatgtatcggaaactctggggatcttgatgagtctgtggctgctgcaatttccgataatgatatagttgctgctgctgttctatctggaaataggaattttgagggacgtgtgcatccattgacacgagctaactatcttgcttcacctccgcttgttgtagcttatgcgcttgctggaacagttgacattgattttgaaaaagaacccaattggaacaggggaaggatgggaaaaaacatatacttccaggatatatggccgtccactgaanaaattgcacaggttgtgcaatccantgtttttgcctgaaatgttcaagaatacctatgaagcaatcaccaaagcaccctatgttggaaccaactgactgttcccngccacactctttattcatgggacccaatccncntacatcttaaccaccntattttaagganatgaccatggcccncctgtccncatggggtgaaaaa

SEQ ID NO: 23 - DNA (cDNA), 810 bp; single-stranded, unknown
  immediate source clone: U-U96
ggcacgagagggccgttcctttttgtgctcgctttgctgcctcctcgttcgtcttttccgcgaatttcaccggccatgaacagccttcggagccttccgacatcctccgctccgccctcgccggtcccgagaggacgttcgagtaggccaccgaccttgaatctgggggtgatacgaccacatcatcttcgtgtaaatgtcgccaggagtgcatgttccaagtcttctgcaagtcctgagaagactggtgctgaagtgcaagatcagaagtctgagatatatagcagtgatatgacaaaagcaatgggtgcagtgctgacatataggcatgagcttggaatgaattataatttcatctacccaggattgattgttggttcttgtctacagaccccatcagatgttgataagcttccnaaagattggggttgaagaccatcttttgcttgcagaaggactcggatctttgaatattttggggttgaaattggtgcaattccntgaatatgccatgcaatgtggtgacattgaacatcttcntgctgaaatcagggattttgatgcatttgatttgaagatganacttcctgcngtanttacnaattatacaaggctaattaatccgaatggtggaatgacttattttcnctgtacanccnggcttggaaaacgcctgcanttgcnttggcatacntgtttttgggttnaaggctatgaactgaatgaaagggccccnctactgcnaaattanccaacttgctccccnaaatttggtgctataaaaantgccaccnttgatatgcttatngg

SEQ ID NO: 24 - DNA (cDNA), 765 bp; single-stranded, unknown
  immediate source clone: U-U103
ggcacgagcttggctgtagtcttcttctcaagtaatcttcgtcttcctgatttgcttggttggtggtcggttgcagggcgaagggatggggatggggaggccgagcggagcttggtcgaccccgtggttggtggtggttcttgtgcattggctcctctgggccaccgagaggaggcgaggggcggtggtggaggcctcccatgtggagtttgcatccctccaatctgttcctgcctccgtcgtcgacaacaggctgaggactgggtatcacttccagcccccgaggaactggatcaacgatccaaatggacccatgtacttcaatggcgtctaccacctcttctaccagtacaaccccaatggctccgtgtggggtaacatcgtgtgggcccactcggtgtcgaccgacctcgtcaactggatagcactcgacccggccatccgccccagcaagccattccacatcaacggatgctggtccggctccgccaccgtcctccccggcaacaggcctgcgatcttctacaccggcatcnacccccancanaaacaactgcanaacnttncntncccaaggatctgtccaaccttactccccnantgggtcaaccccactncaaccccgtgatccccctggcgacgcatcaacnccaccccttccncnaacccnaatacggnttggcgcngtcccngcacccnctggaaacccctngtngggaacnanttgaancngaaggggaaaggcnttctttnccgaacagggat

SEQ ID NO: 25 - DNA (cDNA), 774 bp; single-stranded, unknown
  immediate source clone: U-U93
ggcacgagcggagaangcaatctcgctctctctctactgcatcgcggctctcgtcctcgattacttgagatatggcaggaaagggcgaggggccggcgatcgggatcgatctcggcaccacgtactcgtgcgtcggggtgtggcagcacgaccgggtggagatcatcgccaacgatcaggggaatcgtaccaccccctcctatgtcgccttcaccgactccgagcgccttatcggcgacgctgccaagaaccaggtcgccatgaaccccatcaacaccgtctttgatgcaaagcgtttaattggtaggcgattcagtgattcatctgtccagagtgacatcaagctctggcctttcaaggtcattcctggtccaggtgacaagcccatgattgttgtccaatacaaaggggaggataaacagtttgcagctgaagatatttcttccatggttttgataaagatgaaagaaattgctgaacctacctggggtctgttgtcaagaatgccgtcctcactgttcccgcttacttcaatgactcncaacgcccagccacaaaggatgctggtgtcattgctggtctcnatgttaatganaatcatcaatgancccccagcactgctattgctttatggccttgacaaaangctactattgttggtgaaaaaaatttcctcatcttccatctccggtgggtggccttttganttcnccttcctnaccatccaagaaggtttctttnaagtcaaggccctgctggtganacccancttgg

SEQ ID NO: 26 - DNA (cDNA), 786 bp; single-stranded, unknown
  immediate source clone: U-U125
ggcacgagctagtctcgagtttttttttttttttttttaaaattgaaattagatgcgatttcgaatagcaaacatagttcaggataaaccaacaacattgtaccgaattcgataagcttaggaaactaaattgcgtaaacgaaacacttgcatgaaagcctatataatataaggccgactagggacnaaataacttaatcgacctcctcgatctttggaccagcgccgctgccaccagcaggaggagcatcatcatccataccaccagccatgtcagcacctgctccctggtacatcttggcgatgatgggattgcatatgctctccagctccttcatcttgtcatcgaactcgtcggcttctgccaactggttgccatcgagccattggatggcctgctcgatcgcatcctccgatcttcttcttgtcagcagcagcaagcttggaggcaatcttgtcgtcgttaatggtgttcctcatgttgtnagcatanttttccagancattcttccgattccaccttcctttttatgctcctccatcttcctgacttgttacttctccgcttccctgcaccattttctcaatctcctccttgctgacctaccttgtccttggtgaaggtgatcttgttctctgtccagtggtctntcntcngcagaaanattccagaataccattggggttccaaatgttncaaaaccaaacccggtggattcttgaaggaaacccccccctaagggccccagggtgggaaatccccccaaaaaacccccaaattttccc

SEQ ID NO: 27 - DNA (cDNA), 486 bp; single-stranded, unknown
  immediate source clone: U-U105
ggcacgagcgagttttttttttttttttttttttttttttttttcacaaggnaaccatagacattacaccaaacatagatgtcaacaatacatgccaacgataccattgcttattccttcagacctccgataggacttccacgcacaccaacagagagaactaagacagcaaagcagcggacacatacagcccaaaagggtnngtgacccccgcggaggnggagcnccaggtgaagggtggattccctctgaatgttgtattctgncagggtgcgcccgtcctcnntttgnttcccntctnanatcncccnctgntgatctggtggnaatcccccccttgtcctgtgattcttgctcttgacntttttctcccntgtcnnanctctctncctnctctgggtnttgggtcttcccctgttgnancnntctnttnncntatcatcttgtnctccnnccnccncncctntcntttcctcnnnnggtnna

SEQ ID NO: 28 - DNA (cDNA), 791 bp; single-stranded, unknown
  immediate source clone: U-U116
ggcacgagtgacaatgtcaaggccaagattcaggataaggagggcattccccccgaccagcagaggctcatctttgccggcaagcagctcgaggatggccgcaccttggctgattacaacatccagaaggagtccaccctccaccttgtcctccgcctccgtggtggcatgcaaatcttcgtcaagactttgactgggaagaccatcacccttgaggtgganagctcggacaccatcgacaatgtaaaggccaagattcaggacaaggagggtnttcccccggaccagcaaaggctcatctttgccggcaagcagcttgaggatggccgcaccctggcagattacaacattcagaaggagtctacccttcaccttgtgctganacttaggggtggcatgcanatctttgttaagacnctcncaggggaagaccattaccttggaggtggananctcngacacgattgataatgtcnaggcaaagatccangacaaggangggattccaccggatcagcanangctgatctttgctgggaaacactggangaagggcgcaccctggcagattncnacattccnaaagaatccaccttcacctggtgctccccctccgccggggtccctaacccttttgggctgtatgtntcccctgcctttgctgtcttaattcnctctgttggtgttcgttggaaattccctaatccngaaggttcttnaaaaggaaatnaanccnnttgggttttcccctttggccttttttttgtttgaaaatcctntgtttttggtgt

SEQ ID NO: 29 - DNA (cDNA), 814 bp; single-stranded, unknown
  immediate source clone: U-U120
ggcacnancccgcttcggtagccattattggtgcccgtagagcgagtgctgagatggctttgagggccttcttccccctctgcatcgctctcgtggtgacgctctcacctctgtgtgatgccacttcgccttactacaccatcacaccgcccacccccgtggccaagccgccttcagttgaaccaccaccctaccacggccctccgacnacccaccctaagccaccgagtcatggtggccaacctccgtcccaccatcacccaacaccaatctacggtgcaccccctccgcaacaccaccaccaccaccaacaccaccaccaacctgcaccaccaactcacgcanaacaccctccgtactaccacatgccttccccgccgccgcatggccagcacccgtcaccaccgtcacatgattatcccgtacctcctgctcacaagccccgaactccgccgccggtttacaagtctccaccaccganccaccgtccttaccctccatcgacgccaccccaccatccgaagcacccgccttctcagccgaagccgtcntacaaggcccgccaccatncaagaacatccctgancactctccaccgccgcgtcactatcattctccgtcttcaccaccaccaccaccaccatacaaaataatctcgttttgccatcttctcccgtttgaaaaaaaanncaantgtcctttttatttaaccnaatccanggggtttttnttaanatttnaanaaancnaantnttttcccccccntnttggttnatggcctatgggtttgtttctcttcttggcttttcccgg

SEQ ID NO: 30 - DNA (cDNA), 742 bp; single-stranded, unknown
  immediate source clone: U-U126
ggcacgaggttgtgcctaacagagagagagagagacagaccgatagcctcctcattcactatggcgatccgatcgccagcttcgctgctgttatttgcgtttctgatgcttgcgctcacaggaagactgcaggccgggcgcagctcatgcattggcgtctactggggacaaaacacagacgagggaagcttagcagatgcttgtgccacaggcaactacgaatacgtgaacatcgccacccttttcaagtttggcatgggccaaactccananatcaacctcgccggccactgtgaccctcggaacaacggctgcgcgcgcttaagcagcgaaatccagtcctgccaggagcgtggagtcaaggtgatgctctccatcggaggtggcgggtcttatggcctgagttccaccgaanacgccaaggacgtggcgtcatacctctggcacagtttcttgggtggttctgctgctcgctactctcnacccctcggggatgcggttctggatggcatanacttcaacatccccggagggagcacagaacactatgatgaacttgccgctttcctccnagggctacaacgaacaggaagccggaaccaaaaaagtttcacttgaattgctgctcccncantgtcctttccccgattactggctttggcaacccactccaaaacaaatntcttccnacttccntgttgggttgcnaanttccnttcccaancaaacccctttcntttgc

SEQ ID NO: 31 - DNA (cDNA), 794 bp; single-stranded, unknown
  immediate source clone: U-U129
ggcacgagggcgtggcggagatggggagctggcgggctctgttgcagcggcggctgctgttgctctctgctttggcggtggctgttcgtgtgaaggcactcagcananacnatttcccctccggcttcatttttggcgcaggcacctccgcttatcaggtanaaggtgcagctgcagaggggggaanaacacccagcatttgggacacgtttacgcatgcagggagaactttcgaccagagcaccggagacgtagcagctgatcagtatcacaagtacaaggaagatgtgaanctgatgcatganatgggcttcgatgcttacagattctccatctcctggtccanagttatccccaatggtcgagggcctgtgaatccacaaggcttgcggtactacaacaacctgatcgatgagctcnaaagatatggaatccgagcctcatgtcactctttaccacttcgaccttccgcaagcactggaanacgaatncgccgggcagctgaacccaaagatcgtanangacttcaccgcttacgccaacgttttgcttcancnaatttggggatcnaatctagcactggatcncccatcaatgaaccaacatanatcccgtcctccgccacnaattccgcatcttcncccctggcccctgctctttatcccttncgcctccaactgcnccaagggcnactcccccccattttnaancccatnnnntccnccccccccaattaacccnttcttgctcttcccccttgccttcanccaacccgcccnttttccnaaaaaana

SEQ ID NO: 32 - DNA (cDNA), 799 bp; single-stranded, unknown
  immediate source clone: U-U130
ggcacgagccggcctcaggcatatggtgcggtcgctacacgtgttcaggcaaggccgggagccggccaggatcatcagggaagcactgtcgaaggcgctggtgaagtactaccccttcgcggggcggttcgtggacnatcccgagggcggcggcgaggttcgtgtcgcttgcactggcgagggcgcttggttcgtcnaggccaaggcggactgcagcttggaggacgtgaagtacctcgatctcccgctcatgatccctgaggacgcgctcctgcccaagccctgcccgggactgaaccccctcgacctccctctcatgctgcaggtgacananttcgtgggcggcggattcgtggtcggcctcatctccgtccataccatcgccgacggcctcggcgtcntccanttcatcaacgccgtcgcccgaaatcgcccgtggcctgccnaancccaccgtggancctgcatggtcccgggangtcntacccaacccacctaagctgcctcccggtggcccgcccgtgtttcccctccttcaanctgctccacgccaccgtcgaactatcccctgaccacatccatcacgtcaagtccccacactttgganctcacccggccaacgctgctctaccttccacntccccatccccaacctgtttgnaatcccgcacgccccgccntcnaccttggaacccaaggcntttccaanctttgcaacccttttttcctttccttccccccccaaaanaacttccccccacccttgtttttgcncccaagttttttccttccttgcccc

SEQ ID NO: 33 - DNA (cDNA), 759 bp; single-stranded, unknown
  immediate source clone: U-U43
ggaggttgttgttcatgtccctacttcggggtctcctgtacagattgaatttcaagtaactaatagcagtggctacttggtgcttcattggggtgcaattcataatagaaggaataactggtcacttccttctcgtcatcctgatggaacaaaagtatacaaaaaccgagctctcaggacaccttttaaaaaatctggttccacatcatcagtaaaaatggagattgatgatcctgaaatacaagcagttgagtttctcatatttgacgagtcagaaaacaaatggtttaaacataatggtcagaatttccatgtccagttattaaaacagggctatcaaaatcaaaatgttttggcctctgtaaatccaaatgtttcactgccanaaagaacttgtgcagattcaagcttatcttaggtgggananaaagggtaggcaaacatatacacctgatcaagaaaaggatgagtntgaagcagctanaactgagtttgctanaaaaactaagtnnaagtacttctgtananganctccgatctaaattgacaaaaaacnccactgctggagcggacaaatccgtcaaagttgaaactatcaaaagtgggatccggatgatcttntgcaagttacangcatacttccnatnggaaaaaccgggaaaccatnttatcccccngaaaaacactnatggaattgaagaaaccagggaaanactgcccatgaattaaataaangaacncncctncaaactnagggaa

SEQ ID NO: 34 - DNA (cDNA), 785 bp; single-stranded, unknown
  immediate source clone: U-U70
ggcacgagagagagagagagagagagagagagagagagagagagagagagagagagagagagagtaatcgatttagcgtgcaaagtactgcagcagaagccatggcatccanggcctcagcntcggctgccctcttccttgctctcaaccttgtcttctttgcactcaccagtgcttgtggcaccacttgcacccccgccactaagcccccaccgagctcttcccatggcaaatgccccgtcgacaccctcaagctggcctcctgcgccaacgtgctcaatggcctgatcnnggtcggcgtcggcaagctcccgaaacagccgtgcgagtgctgcnctctgctcgatggcctcgtcgacctcgaggccgccgtgtgcctttgcnctgctatcanggccnatgtccttggcattcatctcaacctccccatcnncttcagccntctcctcaactactgcggtnagaaggctcccaccggtttccagtgcccttgagctccccctgcggatcacatcntcgatcctctttgcccgtggtgaattttnttgagcattcctttgtgtctttacttccctttgtttttggctttttgatgacgaaagtgttgagtgttgatatgaagggccantggcgctttcntgttaatgttacctgtcnttccctttttatttccacnncttgttgantttccctctcgggtttgttaatgaagatcnatgcattcaattnttaatatttctgctgccttccaanaaaaaatannantncctccnngaantcttcct

SEQ ID NO: 35 - DNA (cDNA), 784 bp; single-stranded, unknown
  immediate source clone: U-U16
cggcacgaggaaangacnagcgatggcagcagaattgtctcccacgctgagcaagagcatctttgaaggagccggtggatcttactctacttggtcaggtgccgatctcccccttcttactgatgcaaagctcggcggaggcaagcttgtcctgaaaccactgggcttggcgttgcctcactattccgactcatcgaaagtcggctatgttcttgaaggaagagcggtggtggggctaacactctatggagagaccgagcagaggatactgctgcttgagaagggagatgtggtagcggtggtcatggggagcctcacgtggtggtacaacgaggaggaggactccgacttctccatcgccttcttaggcgataccgcgacagctgtgcgaccgggcgacatcgcctacttcttcttggcaggatccctaggagtgctccatggcttttcgacggaattcctcancagggcctgcggtataagggatgcggaanctgaagagctcttcggaagccaacctggtactctaatcatcncactgcancaaaagctgcctggcctcanaagcatcccgagcttgacngcgaagggatagtcntnaacnctnancgcgttncggcatatatcaatgtnaanagtggtggctgtgctgcgtccgtgacacttnatgaactggccgcgctgggaagatctgttctccgtcnaactcaccanantcnaacctaaccccntncncttccngggttctccttnatgcactntgcncctatttntttcccaangc

SEQ ID NO: 36 - DNA (cDNA), 817 bp; single-stranded, unknown
  immediate source clone: U-U30
cggcacgagatatggaggtatgatcgggagctactgggagctccgctttggtgaacaacacgacggcaagaagggctatggattcaaggagtgaaggccatggtaccgcanaggcgggtcttccgggcgtgcaccgaattttgcatcggatgaaaaccttggtcatcagcatatgggggctgggttccaccaagggaaaagttcgaatgcaagtaccagtgagtcccatgagagggacttgatcatgcagaggtatgatcgaagcagctggaganttggactgctccanagctcatattcgcttaagggagcccgacaagtcagaggacaaggtcgagtaagcgaacgttgctacccaaaatcaagcatcagttanaatggaggtggactcanaggagtgccacggaagacatctctactgattgtgaaggaaagggatacagaggcgaancgacggatantanggccatgggcatggcagcgccatggtaccgcanangcgggacttccgtgcaagtcattgatcccttgctctcacggaggganancgcttggtcctgaaaggggccnaagaagtggacatgcanangcaatctccaattaccgaaacanggctgaagggcanaagccnanaaacttcctnagaacggtgtcaacaatttctcatcnanatanccgtaattaangacttccggtcatgcnanaatncccnaccnangaaccaancaggcntacttgtgctgtnctttgctacccnntgaatngcgccnggttnatngaaaaaacgtccttccccaagcaaccctctntcaaaaan

SEQ ID NO: 37 - DNA (cDNA), 837 bp; single-stranded, unknown
  immediate source clone: U-U40
ttttttttttttttttttgggtatcaggcaaagatggatacttccaggtttgctatttaagaaccatagtcagtcttatgaggagagaccatacgtaatacaatctttaagatgacaaaatccattcctaccaatacaatgaccaagcgaaaggtacaaggataacgggcaaaagactaaattgcaactatataaaattctgctgcattgtagtaaattcaactgtgacgacacgccaccaggatgaagtctatctataaacccacttattgctagatggacggtgtaacagagctaaaacagcaaagtagaataaaagaaactgacaacgcttccgcatgcgctccacatatggagccaggcggaggttgggcaattggaggaaatgacntttctgacggtgtaacataaaanaaatttgtaacaccgtcaagtacgatttcccatccttcgggttgtggcgttctgctacttggcattacgttaacatcgagtgccgacttggcaagacaggaccccagcatcagaagtcagtgtgtcaaccaaggaagaaggcttggccccaaagctggaggcccgtgcgtaactggctgaacccctgcaccggaangggtgaaaaaagcaccattcagaacaggtcncctccgacctcccaattccagttcttccaccntgcttttgatctgtgtccacctttttgttacctattccatcgaagcntanggatttttaaaaatcccgaaggaaaggaanttccnataattggtgcnccaaanaaggaagatggacngaactgttgttggttgctcctgttcctcctttgcaatggattggtcg

SEQ ID NO: 38 - DNA (cDNA), 799 bp; single-stranded, unknown
  immediate source clone: U-U108
ggcacgaggcagctcaatcgaccgactcctcctcctcctcctcctcctcctcttgttcttctcattctaatataattatcgcttttggtgtgtacatcttctataatttgagaggatggaggcaggattgatggcgaaacaagcagcagcggttgcgaagccgaacgcgttccccgcccggagcctagggttcgggagctcggtcaggggcggatccgggaccagcaggatcgggttcgaggcgcccgcgagcgtggcatggaggaagcggtcgattcaggtggcccgccaaggagccattcggtcggaggtggtcgtggaananaaagcatcnccgcccaaaaaggataaggccggtccgggtcggctctacgtgggtctgcccttggacgtggtctccgacggcaacgtcntcaaccacggcaaggccatcgccgccggtctccgcgccctccgccctcctcggcgttgatggantcnaactcccccatctcctggggcgttggcgatggactccggcgaatggtcctcctacctcccccgtcnccgccatggcgcgcgacncccggccttcgcctccgcgtctccctccacctccactgccancgccgccctcgcctccccctccccnaatcggtcgaatccnccgccgctaacaaaccccgaaatcctattcnctgaacgcnccggccgccnccngttgcccaaantngccttnttcctttccnctttgtttnaaaaaaaactccccnttgttccttccaancgggnaggaacccccnaatggaaaccttncaaa

SEQ ID NO: 39 - DNA (cDNA), 786 bp; single-stranded, unknown
  immediate source clone: U-U128
ggcacgaggaaacgtctctccctctctccacttctcacacccccgcccccgactcagcgaatcgacattcttggcgtgtcaaggaaacctttcttttatttgcgaaggaacatgagcaatactgttggacacaccttaccccagcaagctctttaccggccttctatcaaagaaaatcaaagtaaagctcatcaaggagtttctgcaaacttcttgtgtggagttccattaggatctaaggtggaaaatgcagtgtcatacagctctaggtcactgctttcaaccggatccctgggaaagaaactcatcaaaggaatcccaccaaagcaaaatccaagcatagtcactatgacccctcgtgctgtgttggctgcanatcctgcttcagagctaaggagaaaattcaagcttgacacaaattctgaattggaggttgttgttcatgtccctacttcggggtctcctgtncagattgaatttcaagtnactaacagcagtggctacttggtgcttcnttggggtgcaattcataatanaaggaataactggtcacttccttctcgtcatcctgatggaacaaaagtttnccnaaacccgaactctcanganaccttttaaaaaatctggttccccatcntccgtnaaaatggagattgatgatcctgaaataccagcagtngaatttctccttanttttgnaacaaatnctcaaaaaaaccnaaattgggtttttaaaacnattaatgggtcccnaaatttccntgttccnntttttttanaaacaag

SEQ ID NO: 40 - DNA (cDNA), 804 bp; single-stranded, unknown
  immediate source clone: U-D4
ggcacgagagatagctgaagtgacgtgaggtgacttcgattagatcgagagaaatttctttgtgatttttgtggacaattctcaatggctgctgtaacaaantcacaattcatctcgaaaggctcatgcgctggctatgggggtgtcgtcgactcggagccaaggaccttcctaaacagaagggttctacacttgaggaaccaaaccaccgcttacgaaggattgagatctcgaaatgtggttgatttgattcagatgccgtctaatgccaaggtgatttcaaggaaaactgtgaggggaactcagaatcctagtcgtanaccttgggctgttgtcatttgtggaaaagggatgaacatagtctttgtcggcgctganatggctccctggagtaaaaccgganggactcggtgatgttcttggaggactgccaccggccatggctgcaaatggacacagantgatgactatagctccaccctatgatcagtacaaggatgggtgggatacaaatgtcctggctgaattaaaagttggaaatgaaattgaaanaatccgctttcttccactgctataaaaaaagaattgacagggttttcattgatcacctttgtttcttgaaaangtgttgggaaaaactggaagaatgaaattttggtcctntccccggaactgattttnaaaaacancccctaaantttanccttttntncccngccncttttggaaactcccnggatnctggttttttnccacccccnaaaantttcctngaacctntgggggaaantttttttttttttt

SEQ ID NO: 41 - DNA (cDNA), 826 bp; single-stranded, unknown
  immediate source clone: U-D10
ggcacgagagatagctgaagtgacgtgagtggacaattctcaatggctgctgtaacaaagtcacaattcatctcgaaaggctcatgcgctggctatgggggtgtcgtcgactctgagccaaggaccttcctaaacagaagggttctacacttgaggaaccaaaccaccgcttacgaaggattgagatctcgaaatgtggttgatttgattcagatgccgtctaatgccaaggtgatttcgaggaaaactgtgaggggaactcagaatcctagtcgtanaccttgggctgttgtcatttgtggaaaagggatgaacatagtctttgtcggcgctganatggctccctggagtaaaaccggaggactcggtgatgttcttggaggactgccaccggccatggctgcaaatggacacagaagtgatgactatagctccacgctatgatcagtacaaggatgggtgggatacaaatgtcctggctganttaaaagttggaaatgaaattgaaagaatccgcttcttccactgctatnaaanaagaattgacagggttttcattgatcatcctttgttttcttgaaaangtgttgggaaaaactggangaatgatatntggtcctgtcccnggaacggattattnaaaaccacccncttaanatttacccttttgtgcccangcancntttggaanctcccaaggattctggatntttaacnacnnccnaatacttttcctggaaccatttngggganaannttgttntttgttgcnaattaattggcnccctggtcctcctncctgctacttaaaaattttntttcntnt

SEQ ID NO: 42 - DNA (cDNA), 813 bp; single-stranded, unknown
  immediate source clone: U-D13
ggcacgagctgagggagtgagggaaagagatagctgaagtgacgtgaggtgacttcgattagatcgagagaaatttctttgtgatttttgtggacaattctcaatggctgctgtaacaaagtcacaattcatctcgaaaggctcatgcgctggctatgggggtgtcgtcgactcggagccaaggaccttcctaaacagaagggttctacacttgaggaaccaaaccaccgcttacgaaggattgagatctcgaaatgtggttgatttgattcagatgccgtctaatgccaaggtgatttcaagggaaaactgtgaggggaactcagaatcctagtcgtagaccttgggctgttgtcatttgtggaaaagggatgaacatagtctttgtcggcgctganatggctccctgggagtaaaaccggaggactcggtgatgttcttggaggactgccaccgggccatggctgcaaatggacacagaagtgatgactatagctccacgctatgatcagtacaaggatgggtgggatncaaatgtcctggctgaattaaaagttgggaaatgaanttgaaagaatccgcttcttccactgctataaaanaagaattgacagggttttcattgatcacctttgttcttgaaaaggtgtggggaaaaactggaagaatgatatttggtcctgtcccnggactgatttnaaaacaccacctaaaattaccttttgtnccngcnnctttggaaccncccnggatctngatnttancacccccaantcttatcctggaacttttggggnaaantttgtntttgtt

SEQ ID NO: 43 - DNA (cDNA), 805 bp; single-stranded, unknown
  immediate source clone: U-D66
ggcacgagaagagatagctgaagtgacgtgagtggacaattctcaatggctgctgtaacaaagtcacaattcatctcgaaaggctcatgcgctggctatgggggtgtcgtcgactcggagccaaggaccttcctaaacagaagggttctacacttgaggaaccanaccaccgcttacgaaggattganatctcgaaatgtggttgatttgattcagatgccgtctaatgccaaggtgatttcaaggaaaactgtgaggggaactcacaatcctagtcgtanaccttgggctgttgtcatttgtggaaaagggatgaacatagtctttgtcggcgctgagatggctccctggantaaaaccggaggactcggtgatgttcttggaggactgccaccggccatggctgcaaatggacacacagtgatgactatagctccacgctatgatcagtacaaggatgggtgggatacaaatgtcctggctganttaaaanttggaaatgaaattgaaaagantccgcttcttccactgctataaaanaagaattgacagggttttcattgatcatcctttgtttcttgaaaaagtgtggggaaaaactgggaagaatgatatntggtcctgtcccnggaactgattataaaacaccagctaanattaccttttgttgccagcaactttggaaccccnaggattctggatnttacaacacnaatactattctggaacatttggggaaaaattgtttttgttnccaatgantggcncactggtcccntccctncctacttaaaaanttttttcc

SEQ ID NO: 44 - DNA (cDNA), 790 bp; single-stranded, unknown
  immediate source clone: U-D111
ggcacgagaagagatagctgaagtgacgtgagtggacaattctcaatggctgctgtaacaaagtcacaattcatctcgaaaggctcatgcgctggctatgggggtgtcgtcgactctgagccaaggaccttcctaaacagaagggttctacacttgaggaaccaaaccaccgcttacgaaggattgagatctcgaaatgtggttgatttgattcagatgccgtctaatgccaaggtgatttcgaggaaaactgtgaggggaactcagaatcctagtcgtagaccttgggctgttgtcatttgtggaaaagggatgaacatagtctttgtcggcgctganatggctccctggagtaaaaccggaggactcggtgatgttcttggaggactgccaccggccatggctgcaaatggacacagagtgatgactatagctccacgctatgatcagtacaaggatgggtgggatacaaatgtcctggctgagttaaaagttggaaatgaaattgaaagantccgcttcttccactgctataaaagaaganttgacagggttttcattgatcatcctttgtttcttganaaggtgtggggaaaaactggaagaatgatatatggtccctgtcncaggaacngattatgaanacaaccagctaagattaaccttttgttgccagcactttgggaaccncaaggatcctggatcttaacaacncaantctattctggaccatatggggacaaattgttttgttgcaaatgatggcacctggtccnntccatgctactta

SEQ ID NO: 45 - DNA (cDNA), 813 bp; single-stranded, unknown
  immediate source clone: U-D112
tttttttttttttttttttggagcataatagcaatttattcaggaggatcaaactcaaacactataacatagacagtagtcactttcagggagtggccacattttcctttgcaagaggagctatttcatcagcatcgatgccggcttcactaccagcagctcctaagcttagganaaattgctcccacttcttagcaggtcccttccaggagaggtcttgagtcatgcagttctgtatcatctcagcaaatgcaggtgtgccgtaaactttaagggcccttttcactgtctgaatgactttttgtacatcatctttatcgacaacatcacaatcaggattgaagggacccatatgaaaccctgtgatgccttctatgacagtgtcagcaagtccaccagttgttgcgcacatggggggaattccatatcgcatgccctgaaagctggataaggccacaaggttcgaatctgctggtaagancaagaagatctgctccagccatgatttcatganccaaaggtgcattaaacttcatatgtgctctcactttattgggaaacatatcttcaagttgtgcaagctgacgctccaacancttctcccantaccaagcactatcacttgaacattttcatcaatgaattcaggaataactgcngcgaaaatatccgaacctttctgctctctaatctccctataaaggntataacagggatttccngtcaacaggttaaccaaattcaacttgttaggnttcccttaatccaaaggtttgcacccnncccatttgttgctcntaattganaaaaatt

SEQ ID NO: 46 - DNA (cDNA), 808 bp; single-stranded, unknown
  immediate source clone: U-D39
ggcacgagcgagttttttttttttttttttgctgtgatgatattatgcatcttattagttggttgcaccacttctattacatcactcaacagtattagcccaaganaaggctaataccttcncatcactcttgacatcttatttgtattgtagganactagtatatatatctatatgcgtggttgcctgcacnagggcggcttattagtggcgtcagtgatctcaagggcagaanacaaccctgtanttggtgccgccggggcaggtgaaggtgctcgtctgatcgtccttgggatanctgtaggcgtcggggcagtttctcttgaanaaccgggagtattcggtcgggctgcagctgccggagttgcaacagtactggtccgtcttgaacacggtgcaggggttgttgcanccgcccggcgccttcancgccccggggcactgcccgttgatntccgccgcgcaccggatgccgcggcanccgcctgacntggggctnaatcccattggcacgttgaaaccgtcnaccaagggaaatttcaaaaaatccaagttgttgaactggttgaacgccaactccnccagggtnttgggccggttgccntaccccgtgcatnacaacaccccgccgcantccccttctggcaccgccgcntccgctcccctcaaaagaacacccgtgcggcccaaaatccggncccccggtggttccccctttcnctttaanggtccccaatgcccctgntttaactgnctcccccccangcncggccccggcccacnctttnttaaaacatcngtnccnaat

SEQ ID NO: 47 - DNA (cDNA), 809 bp; single-stranded, unknown
  immediate source clone: U-D50
ggcacgagtgtgcctaacagagaaagagagaccgatagcctcctcattcactatggcgatccgatcgccagcttcgctgctgttatttgcgtttctgatgcttgcgctcacaggaagactgcaggccgggcgcagctcgtgcattggcgtctactggggacaaaacacagacgagggaagcttagcagatgcttgtgccacaggcaactacgaatacgtgaacatcgccacccttttcaagtttggcatgggccaaactccanagatcaacctcgccggccactgtgaccctcggaacaacggctgcgcgcgcttaagcagagaaatccagtcctgccaggagcgtggagtcacggtgatgctctccatcggaggtggcgggtcttatggcctgagttccaccgaagacgccaaggacgtggcgtcatacctctggcacagtttcttgggtggttctgctgctcgctactctcgacccctcggggatgcggttctggatggcatanacttcaacatcnccggagggagcacagaacactatgatgaacttgccgctttcctcaagggctacnacgaacaggaagccggaacgaaaaaanttttacttgaatgctgctcccncantgtcctttcccggattactggcttggcacccactcanaaaanatctcttccnacttccntgtgggttgcanttcctccaanaacccttccttgccntttctccccaaaacctatccatcttgcaattcctttcacaaattgggtcntntccntcccngccccnaaaactttttccctngggctcc

SEQ ID NO: 48 - DNA (cDNA), 809 bp; single-stranded, unknown
  immediate source clone: U-D86
ggcacgagccacaagcttgcctttggttgtgcctaacagagagagagagagagacagaccgatagcctcctcattcactatggcgatccgatcgccagcttcgctgctgttatttgcgtttctgatgcttgcgctcacaggaagactgcaggcccggcgcaactcatgcattggcgtctactggggacaaaagacagacgagggaagcttagcagatgcttgtgccacaggcaactacgaatacgtgaacatcgccacccttttcaagtttggcatgggccaaactccagagatcaacctcgctagccactgtgaccctcagaacaacggctgcgcgcgcttaagcagcgaaatccagtcctgccaggagcgtggagtcaaggtgatgctctccatcggaggtggcgggtcttatggcctgagttccaccgaagacgccaaggacgtggcgtcatacctctggcacagtttcttgggtggttctgctgctcgctactctcgacccctcggggatgcggttctggatggcataaacttcaacatcnccggagggagcacagaacactatgatgaacttgccgctttcctcaagggctacaacgancaggaagccggaacgaaaaaagttcacttgaatgctgctccccantgtctttcccggattactggcttggcaacgcnctccaaaacaaatctcttccacttcctgtgggtgcantcctccacaaaccttcntgcattctccccaaacctatcaatcttgcnaaatgcgttcaacaattngggtcttttcatccccgcccaaaact

SEQ ID NO: 49 - DNA (cDNA), 785 bp; single-stranded, unknown
  immediate source clone: U-D90
ggcacgaggagagaccgatagcctcctcattcactatggcgatccgatcgccagcttcgctgctgttatttgcgtttctgatgcttgcgctcacaggaagactgcaggccgggcgcagctcgtgcattggcgtctactggggacaaaacacagacgagggaagcttagcagatgcttgtgccacaggcaactacgaatacgtgaacatcgccacccttttcaagtttggcatgggccaaactccagagatcaacctcgccggccactgtgaccctcggaacaacggctgcgcgcgcttaagcagagaaatccagtcctgccaggagcgtggagtcacggtgatgctctccatcggaggtggcgggtcttatggcctgagttccaccgaagacgccaaggacgtggcgtcatacctctggcacagtttcttgggtggttctgctgctcgctactctcgacccctcggggatgcggttctggatggcatanacttcaacatcgccggagggagcacagaacactatgatgaacttgccgctttcctcaaggcctacaacgagcaggaagccggaacgaaaaaagtttacttgaattgctgctccgcantatcctttccngattactggcttggcaacncnctccaaaaaanatctcttccacttcctgttgggtgcagttcttccaanaacccnttcntgccatttctccccaaaacgctttccatctttgcaaatgcctttcaacaattggggtcttgttccnnccctgccccaaaaactgttcct

SEQ ID NO: 50 - DNA (cDNA), 813 bp; single-stranded, unknown
  immediate source clone: U-D93
ggcacgagcgatagcctcctcattcactatggcgatccgatcgccaacttcgctgctgttatttgcgttcctgatgcttgcgctcacgggaagactgcaggcccggcccagctcatgcattggcgtctactggggacaaaacaccgacgagggaagcttagcagatgcttgtgccacaggcaactacgattacgtgaacatcgccacccttttcaagtttggcatgggccaaactccagagatcaacctcgccggccactgtgaccctcggaacaacggctgcgcgcgcttgagcagcgaaatccagtcctgccaggagcgtggcgtcaaggtgatgctctccatcggaggtggcgggtcttatggcctgagttccaccgaagacgccaagggaagtagcgtcatacctctggcacagtttcttgggtggttctgctgctcgctactcgagacccctcggggatgcggaactggatggcatanacttcaacatcgccggagggagaacagaacactatgatgaacttgccgctttcctcaaggcctacaacgancaggaagccggaacnaaaaaagttcacttgaattgctgctccgcagtgtcctttcccggattactggcttggcaacgcactcagaacanatctcttcnacttcctgtgggtgcaatttcctccacaanccttccntgccatttctcccaaaaagctatcnatcttgcaaatgcgttcnacaattgggtcttttccancctgcncaaaactgttccttgggctcccgctgcccctgaaggtgcnccaattggtggctnct

SEQ ID NO: 51 - DNA (cDNA), 819 bp; single-stranded, unknown
  immediate source clone: U-D61
ggcactcaggaactccaacatccaagtcctgttggatgtcccccgatccgacgtgcagtcactggcctccaatccttcggccgccggcgactggatccggaggaacgtcgtcgcctactggcccagcgtctcctttcgatacatagctgtcggaaacgagctgatccccggatcggatctggcgcagtacatcctccccgccatgcgcaacatctacaatgctttgtcctcggctggcctgcaaaaccagatcaaggtctcgaccgcggtcgacacgggcgtcctcggcacgtcctaccctccctccgccggcgccttctcctccgccgcccaggcgtacctgagccccatcgtgcagttcttggcgagtaacggagcgccgctcctggtcaatgtgtacccttattttagctacaccggcaacccgggacagatctcgctgccctacgccctgttcacggcctccggcgtcgtcgtgcaggatgggcgattcanctatcagaacctgttcgacgccatcgtcgacgcggtcttcgcggcgcctggaaaaaatgggaagggcgaacgtggcggtggtggtgtccgaaaacgggtggccgtccgcgggcggaagaacccnaaacnaacaccancaacnccgccggaantttcaaccanaacttgatcaggcatgttnggcggaagaaccccaaggaaaaccagggaaggaaatcaaggcttcntnttccaaaantttccaccaaaaaccnaaaggtgganggatccaacaaaactttggcctgttttatcccaannaacancccntcttaccnaataact

SEQ ID NO: 52 - DNA (cDNA), 794 bp; single-stranded, unknown
  immediate source clone: U-D63
ggcacgagggattataaaaagaaactcgcactcagccagattgactcaagcatgaaggttgcatctttcgaatgttccttttttttccaacatcttttgaatgttacatgacaatcatgacgccgtgcatacaatgatacctctgatagggttgaatgcacaaaccactcatcgtgctatattgcaagtgcttatgtttgtattcattttcttataatttcaatgtgagagttaccttaggttcatattanatttatataatattgggcatagatttttttgtcattgtttgtgatctgcaatttcattaggtcatcgaccatattatggtaaatttaatttactaaattatatcttgagtttattgctttgcatattttgtgctggaaaccatgccaatccaactcctttgcaaaacttaagataattaaaggagatattgaggatgccgactgctttaggttatttgcattanacaataaacgcccgtgaaatggaaatcatgcaaagctttgcatggagaatatgttnatagagatagacatgaagacatgaatatgctttcttgatgaacaacgatgttcacntgtttactgcatgcatgatacatccaatgttctaggcttgttggcaatcattttattcnaaaaattgtctgtttctctnccnccataaggttagcttgtggaaaatgttcaactttgggcncanatgatccgttttagcaaaatcccnccattcctatttttttccggaatccnnttggnaaaanatcctnacatactatttccaca

SEQ ID NO: 53 - DNA (cDNA), 798 bp; single-stranded, unknown
  immediate source clone: U-D65
ggcacgaggcccgtagagcgagtgctgagatggctttgagggccttcttccccctctgcatcgctctcgtggtgacgctctcacctctgtgtgatgccacttcgccttactacaccatcacaccgcccacccccgtggccaagccgccttcagttgaaccaccaccctaccacggccctccgacgacccaccctaagccaccgagtcatggtggccaacctccgtcccaccatcacccaacaccaatctacggtgcaccccctccgcaacaccaccaccaccaccaacaccaccaccaacctgcaccaccaactcacgcagaacaccctccgtactaccacatgccttccccgccgccgcatggccagcacccgtcaccaccgtcacatgattatcccgtacctcctgctcacaagccccgaactccgccgccggtttacaagtctccaccaccgacccaccgtccttaccctccatcgacgccaccccaccatccgacgcacccgccttctcagccgacgccgtcatacaaggccccgccaccatacaagaacatccctgancatctccaccgccgcgtcactatcattctccgtcttcaccaccancacaaccaccatncaaatagtctcgtttgcatctctccgttgaanatgaaccaatgtcntttaataacgatcagggtttcaaataaaaacnaatttccgccattgtaatgctatggttgttctctctgcttcccggggaaagtttcttgggtcatnttaaccncctcctaatgttcngctcttnntanaa

SEQ ID NO: 54 - DNA (cDNA), 824 bp; single-stranded, unknown
  immediate source clone: U-D84
ggcacgagcgcccacccccgtggccaagccgccttcagttgaaccaccaccctaccacggccctccgacgacccaccctaagccaccgagtcatggtggccaacctccgtcccaccatcacccaacaccaatctacggtgcaccccctccgcaacaccaccaccaccaccaacaccaccaccaacctgcaccaccaactcacgcagaacaccctccgtactaccacatgccttccccgccgccgcatggccagcacccgtcaccaccgtcacatgattatcccgtacctcctgctcacaagccccgaactccgccgccggtttacaagtctccaccaccgacccaccgtccttaccctccatcgacgccaccccaccatccgacgcacccgccttctcagccgacgccgtcatacaaggccccgccancatncnagaccatcccttgagcacttctccancgnttgcgtttattatcattctccgtcttcaccaccaccaccaccaccatacaaatagtctcgtttgccatctctccgttgaagatgacgcagtgtcgttttagtagcgatcagggttaccaataagaacgatgttgccgccattgtaatggctatggttgttctctctgcttttccgggggaaggttctttgggttcatgttaaacctctctcttaaatgttcatgcatcttattataaacnaaaattggccatttnnnnnntntnntnnnnnnttnnnnnnnnaaaactcnaaaaatatttttaaaaacgggcggggggcccatcnattttccnncccgggtnggggntcccagnttattnt

SEQ ID NO: 55 - DNA (cDNA), 797 bp; single-stranded, unknown
  immediate source clone: U-D75
ggcacgagggttacgatcgtcatgttgggggtgttcagcggggnaggtggtggaggtgccggcggagctggtggccgccggcagcaggacgccgtcccctaagacacgagcgtcggagctgatcaaccgattcctcgggagcttcgctcccgcggtgtcgatccagatcggggacttgggacacctcgcctactcccacgccaaccagtcccccttcgctcccaggttgtttgcagcgaaggacganatttactgcctcttcaagggagtgctgaccaacctgggcagcttgaggcagcagtatgggctttccaagagtgccgacnaggtggtgctggtcatcgaagcctacaaggccctccgtgaccgagctccctatcctcccagcttcatgctcgcacaccttactggcaacttcgccttcgtgctcttcgacaagtccacatcatccatccttgttgcatctgacccanatggaanantacccttgttctgggggatcactgcanatggatgccttgcctttgctgacnatctanacttgctgaagggatcgtgcgggaagtcacttgcaccattccctgaaggatgttactattccaatgccttggggggcctgaaaanctatgaaaaccccaacacaaggtgaactgctgtttcttgaanatgaagaanaattntttgtgccnctttcaaggtggaangatctgccnttcttgcggnaaccccctaatcaaggaacatcttccaatgtttggggcnaatgatcctcttaaaatcctaaaattcttggaatt

SEQ ID NO: 56 - DNA (cDNA), 804 bp; single-stranded, unknown
  immediate source clone: U-D83
ggcacgagcttccacacaggtgagggtaagtcggttacgatcgtcatgttgggggtgttcagcggggaggtggtggaggtgccggcggagctggtggccgccggcagcaggacgccgtcccctaagacacgggcgtcggagctgatcaaccgattcctcgggagcttcgctcccgcggtgtcgatccagatcggggacttgggacacctcgcctactcccacgccaaccagtcccccttcgctcccaggttgtttgcagcgaaggacganatttactgcctcttcaagggagtgctgaccaacctgggcagcttgaggcagcagtatgggctttccaagagtgccgacnaggtggtgctggtcatcgaagcctacaaggccctccgtgaccganctccctatcctcccagcttcatgctcgcacaccttattggcaacttcgccttcgtgctcttcgacaagtccacatcatccatccttgtttgcatctgacccanatggaaaaatgcccttgttctgggggatcactgcanatggatgccttgcctttgctgacnatctanacttgctgaagggatcgtgcgggaantcacttgcacattccctgaangatgttactattccaatgccttgggggggctgaaaanctatgaaaacccaancacaaggtgactgctgtcttgaanataaagaaaaattttttgtgcccctttcaaggttgaangatctgcattcttgcggcaccccctnatcnaggaacntcntnccaatgttggggcaaatatcctctttaaaancataaaa

SEQ ID NO: 57 - DNA (cDNA), 803 bp; single-stranded, unknown
  immediate source clone: U-D64
ggcacgagggattataaaaagaaactcgcactcagccagattgactcaagcatgaaggttgcatctttcgaatgttccttttttttccaacatcttttgaatgttacatgacaatcatgacgccgtgcatacaatgatacctctgatagggttgaatgcacaaaccactcatcgtgctatattgcaagtgcttatgtttgtattcattttcttataatttcaatgtgagagttaccttaggttcatattagatttatataatattgggcatagatttttttgtcattgtttgtgatctgcaatttcattaggtcatcgaccatattatggtaaatttaatttactaaattatatcttgagtttattgctttgcatattttgtgctggaaaccatgccaatccaactcctttgcaaaacttaagataattaaaggagatattgaggatgccgactgctttaggttatttgcattanacaataaacgcccgtgaaatggaaatcatgcnaagctttgcatggagaatatgttnataganatagacatgaagacatgaatatgctttcttgatgaacaacgaagttcacntgtttactgcatgcatgatacatccaatgttctaggcttgttaggcaacctttatccaaaaaattgtctgtactctcccccataaggtaagcctgtggaaaatgttcactttgggccnnatgatcagtttanccgaaaatcccccttcnttatttgttttctgaaacncnttggaaananattccttacatacctttttcacnnanatnttgaacc

(19) United States
(12) Patent Application Publication
(10) Pub. No.: US 2002/0120960 A1
(43) Pub. Date: Aug. 29, 2002

(54) Genetic control of fruit ripening

(76) Inventors: Colin Roger Bird, Bracknell (GB); Graham Barron Seymour, Wellesbourne (GB); Rosybel De Jesus Medina Suarez, Brentwood (GB)

Correspondence Address: HALE AND DORR, LLP, 60 STATE STREET, BOSTON, MA, 02109

(21) Appl. No.: 09/966,881

(22) Filed: Sep. 28, 2001

Related U.S. Application Data
(63) Continuation of application No. 09/242,860, filed on Mar. 29, 1999, now abandoned.
Application No. 09/242,860 is a 371 of international application No. PCT/GB97/02424, filed on Sep. 8, 1997.

(30) Foreign Application Priority Data
Sep. 10, 1996 (GB) 9618862.8
Apr. 25, 1997 (GB) 9708366.1

(51) Int. Cl. A01H 5/00

(52) U.S. Cl. 800/287; 800/290

(57) ABSTRACT

A method of modulating the ripening and/or senescence characteristics in plants of the genus Musa comprises transforming plants with one or more sequences obtainable from the deposited cDNA library having the accession number 40184, regenerating said plants and selecting from the population of transformants those plants having modulated and/or tissue senescence characteristics.

This invention relates generally to the modification of a plant phenotype by the regulation of plant gene expression. More specifically it relates to the modulation of the ripening and/or tissue senescence characteristics and plants derived therefrom. One suitable application of the present invention is the modulation of ripening and/or senescence processes in plants of the genus Musa (referred to herein as banana).

Two principal methods for the control of expression are known, viz.: overexpression and underexpression. Overexpression may be achieved by insertion of one or more than one extra copies of the selected gene. It is, however, not unknown for plants or their progeny, originally transformed with one or more than one extra copy of a nucleotide sequence, to exhibit the effects of underexpression as well as overexpression.

For underexpression, often referred to as “gene silencing”, there are two principle methods which are commonly referred to in the art as “antisense downregulation” and “sense downregulation (also referred to as “cosuppression”). Both of these methods lead to an inhibition of expression of the target gene. Other lesser used methods involve modification of the genetic control elements, the promoter and control sequences, to achieve greater or lesser expression of an inserted gene.

There is no reason to doubt the operability of these methods: they are well-established, used routinely in laboratories around the world and products in which they have been used are on the market.

Gene control by any of these methods requires the insertion of a selected gene or genes into plant material which can be regenerated into plants. This transformation process can be performed via a number of methods, for example: the Agrobacterium-mediated transformation method.

In the microparticle bombardment method, microparticles of dense material, usually gold or tungsten, are fired at high velocity at the target cells where they penetrate the cells, opening an aperture in the cell wall through which DNA may enter. The DNA may be coated on to the microparticles or may be added to the culture medium.

In microinjection, the DNA is inserted by injection into individual cells via an ultrafine hollow needle.

Another method, viz. fibre-mediated transformation, applicable to both monocots and dicots, involves creating a suspension of the target cells in a liquid, adding microscopic needle-like material, such as silicon carbide or silicon nitride “whiskers”, and agitating so that the cells and whiskers collide and DNA present in the liquid enters the cell.

In summary, then, the requirements for both sense and antisense technology are known and the methods by which the required sequences may be introduced are known. What remains, then is to identify genes whose regulation will be expected to have a desired effect, isolate them or isolate a fragment of sufficiently effective length, construct a chimeric gene in which the effective fragment is inserted between promoter and termination signals, and insert the construct into cells of the target plant species by transformation. Whole plants may then be regenerated from the transformed cells.

Bananas are a globally important fruit crop. They are not only a popular dessert fruit, but represent a vital carbohydrate staple in the tropics with as many as 100 million people subsisting on bananas and plantains as their main energy source. The cultivated dessert banana is commonly triploid, parthenocarpic and belongs to the Musa AAA genome group, eg. Cavendish subtypes. Bananas are climacteric fruits and ripening is regulated by ethylene produced by the fruit and involves numerous biochemical changes including the conversion of starch to sugars, cell wall disassembly, synthesis of volatile compounds, changes in phenolic constituents and degradation of chlorophyll in the peel. The conversion of starch to sugars is particularly striking, where starch accounts for 20-25% of the fresh weight of the unripe fruit and depending on the genetic background, can be converted almost entirely to sugars.

The triploid nature of the cultivated dessert banana crop has hampered conventional methods of breeding for improved characteristics. As a result of this an enormous pool of genetic resources for enhancing postharvest characteristics of the fruit has remained untapped.

According to the present invention there is provided a method of modulating the ripening or tissue senescence process in plants of the genus Musa comprising inserting into plant material at least one polynucleotide sequence selected from the sequences depicted as SEQ ID-Nos. 1-57, regenerating said plant material and selecting from the transformed regenerants, plants with modulated ripening or tissue senescence characteristics. The said polynucleotide may be obtained from the cDNA library having the NCIMB Accession Number 40814.

Further according to the present invention is a method of modulating the ripening or tissue senescence process in plants of the genus Musa comprising inserting into plant material at least one polynucleotide sequence or a fragment thereof, obtainable by hybridisation, from the cDNA library having the NCIMB Accession Number 40814, by the use of at least one of the sequences depicted as SEQ ID Nos 1-57 as oligonucleotide probes, said hybridisation being conducted at a temperature from 60° C. to 65° C. in 0.3 strength citrate buffered saline containing 0.1% SDS followed by rinsing at the same temperature with 0.3 strength citrate buffered saline containing 0.1% SDS, regenerating said plant material and selecting from the transformed regenerants, plants with modulated ripening or tissue senescence characteristics. The invention further provides a method as described above wherein the said polynucleotide is capable of modulating the production of pectate lyase and more specifically the polynucleotide comprises at least one of the sequences depicted in the sequence listings as SEQ-ID-Nos. 13-18.

A preferred method for inserting the said polynucleotides into plant material according to the method of the present invention, may be selected from the group comprising the Agrobacterium, microparticle bombardment, fibre mediated or direct insertion methods. The invention further provides plants, their progeny and seed and material obtained from said plants, produced according to the method of the present invention. It is preferred that the said plants, their progeny and seed and material obtained from said plants are derived from plants of the genus Musa.

The present invention also provides a vector functional in plants comprising a promoter region which is operable in plant cells, at least one of the polynucleotide sequences described above and a transcription termination sequence. The promoter may be constitutive, developmentally regulated or switchable. In addition to this the promoter may also be tissue specific or organ specific.

Further provided is a banana produced via the preceding method, having altered fruit characteristics when compared with a banana which is not transformed with at least one of the polynucleotide sequences described above.

The present invention also provides an improved method of controlling plant pathogens comprising the application of an anti-pathogenic agent to plants, characterised in that plants to which the said agent is applied, are plants according to the present invention.

The gene sequences of the present invention may be synthesised ab initio, using the sequence data in the sequence listing provided herewith, or isolated from a library using the standard techniques know within the art. The sequences depicted in the sequence listing or parts thereof may also be used to create oligonucleotide probes for the purposes of isolating from the library those polynucleotides which are capable of producing the desired proteins. To assist the isolation of these polynucleotides we have deposited with the National Collection of Industrial & Marine Bacteria, St. Machar Drive, Aberdeen, UK, a cDNA library of the banana peel ripening related genes. The library was deposited on Jul. 9, 1996 and has the NCIMB Accession Number 40814.

Thus, this invention is based on the identification of genes which encode proteins involved in banana ripening-related processes, specifically within banana pulp. DNA sequences according to the sequence listing or those sequences obtainable from the deposited library, may be used in the process of modifying the plant ripening characteristics of plants and/or fruit.

By virtue of this invention banana plants can be generated which, amongst other phenotypic modifications, may have one or more of the following fruit characteristics: improved resistance to damage during harvest, packaging and transportation due to slowing of the ripening and over-ripening processes; longer shelf life and better storage characteristics due to reduced activity of degradative pathways (e.g. cell wall hydrolysis); improved processing characteristics due to changed activity of proteins/enzymes contributing to factors such as: viscosity, solids, pH, elasticity; improved flavour and aroma at the point of sale due to modification of the sugar/acid balance and other flavour and aroma components responsible for characteristics of the ripe fruit; modified colour due to changes in activity of enzymes involved in the pathways of pigment biosynthesis (e.g. lycopene, β-carotene, chalcones and anthocyanins); increased resistance to post-harvest pathogens such as fungi.

The activity of the ripening-related proteins may be either increased or reduced depending on the characteristics desired for the modified plant part (fruit, leaf, flower, etc). The levels of protein may be increased: for example, by incorporation of additional genes. The additional genes may be designed to give either the same or different spatial and temporal patterns of expression in the fruit. “Antisense” or “partial sense” or other techniques may be used to reduce the expression of ripening-related protein.

The activity of each ripening-related protein or enzyme may be modified either individually or in combination with modification of the activity of one or more other ripening-related proteins/enzymes. In addition, the activities of the ripening-related proteins/enzymes may be modified in combination with modification of the activity of other enzymes involved in fruit ripening or related processes.

DNA constructs according to the invention for gene silencing, may comprise a base sequence at least 10 bases (preferably at least 35 bases) in length for transcription into RNA. There is no theoretical upper limit to the base sequence, it may be as long as the relevant mRNA produced by the cell but for convenience it will generally be found suitable to use sequences between 100 and 1000 bases in length. When using genomic DNA as the source of a base sequence for transcription it is possible to use either intron or exon regions or a combination of both.

As a source of the DNA base sequence for transcription, a suitable cDNA or genomic DNA or synthetic polynucleotide may be used. The isolation of suitable ripening-related sequences is described above; it is convenient to use DNA sequences derived from the ripening-related clones deposited at NCIMB in Aberdeen. Sequences coding for the whole, or substantially the whole, of the appropriate ripening-related protein may thus be obtained. Suitable lengths of this DNA sequence may be cut out for use by means of restriction enzymes. The preparation of such constructs is described in more detail below.

Constructs suitable for expression of the appropriate ripening-related sequence in banana cells, may be produced using a cDNA sequence selected from the deposited library having the NCIMB Accession Number 40814 or the gene sequence as found in the chromosome of the banana plant. Recombinant DNA constructs may be made using standard techniques. In this specification modulation means an increase or decrease of the desired effect. More specifically “modulation of the ripening or tissue senescence process in plants of the genus Musa” means an increase or decrease in production of a ripening related protein resulting from the method as described above. For example, where an increased ripening related protein is desired, plants may be transformed according to the method as described above and those plants exhibiting the desired effect may be selected from the population of transformants. Furthermore, it may be desirable to provide a plant with modulated ripening or tissue senescence characteristics by increasing the production of one protein and decreasing the production of another protein in the same plant. For example, a banana fruit, modified using the present method, and having decreased levels of the enzyme pectate lyase would be beneficial because pulp softening would require a substantially longer time when compared with a control. In addition to this and by increasing the levels of another ripening related protein such as an antifungal protein in the same fruit using the present method, would complement the extended life of the banana pulp with increased resistance to disease. Increase in production of a ripening related protein includes inserting into plant material one or more copies of any of the polynucleotides described above, wherein the said polynucleotides must be capable of producing a protein and thereby increasing protein levels when compared with a control plant.

Decrease in production of a ripening related protein includes inserting into plant material one or more copies of any of the polynucleotides described above, wherein the said polynucleotides must be capable of producing an mRNA which is capable of interfering with endogenous mRNA to such a degree that the levels of translated protein are reduced when compared with a control plant.

Ripening process of plants means the process of maturing or developing.

Senescence means the progressive deterioration in function of cells, tissues, organs etc., related to the period of time since that function commenced.

Control plant means a comparable plant used for the purposes of determining modulation of the ripening or tissue senescence process effect in plants. Specifically, in plant transformations the control plant is usually of the same species and variety as the material used before the transformation process and is grown in the same conditions, (usually with the transformant selection step modified in some way on the part of the control plant), as the transformed plants. More specifically the control plant may comprise an untransformed control plant or a transformed control plant providing it has not already been transformed with the same polynucleotide sequence as the plant material to be transformed.

“Plant material” includes plant cells and any other type of plant regenerable material.

The following examples further illustrate the invention but are not to be construed to limit the scope thereof:

TABLE 1. Is a list of clones isolated from banana pulp and the corresponding sequence identity number as provided in the sequence listing herein. The table also illustrates the approximate clone size, the percentage identity and, where relevant, nucleotide similarity with published sequences, based on the results obtained from comparisons with the EMBL sequence database. The table also provides, where relevant, the gene identity of those published sequences and their database accession numbers.

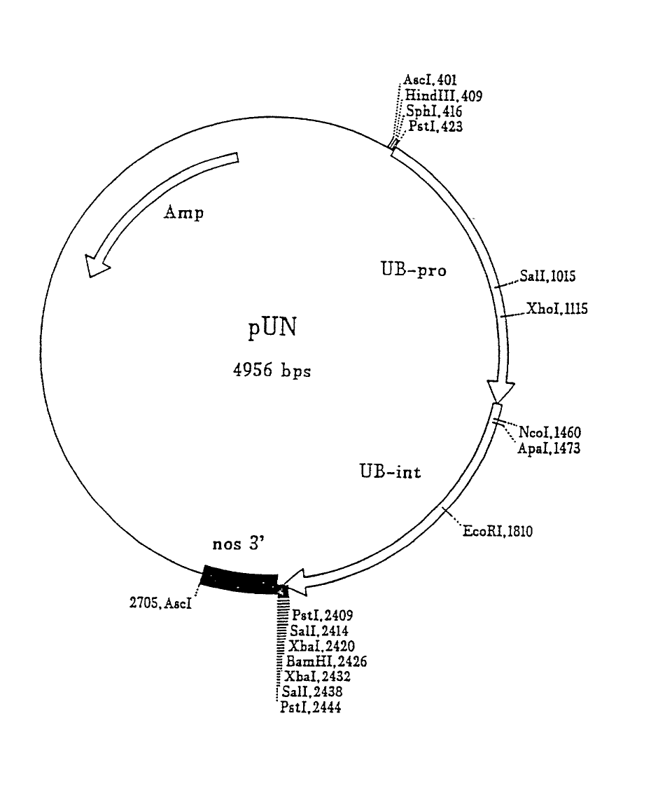

FIG. 1. Plant transformation vector pUN, containing the UBI polyubiquitin promoter.

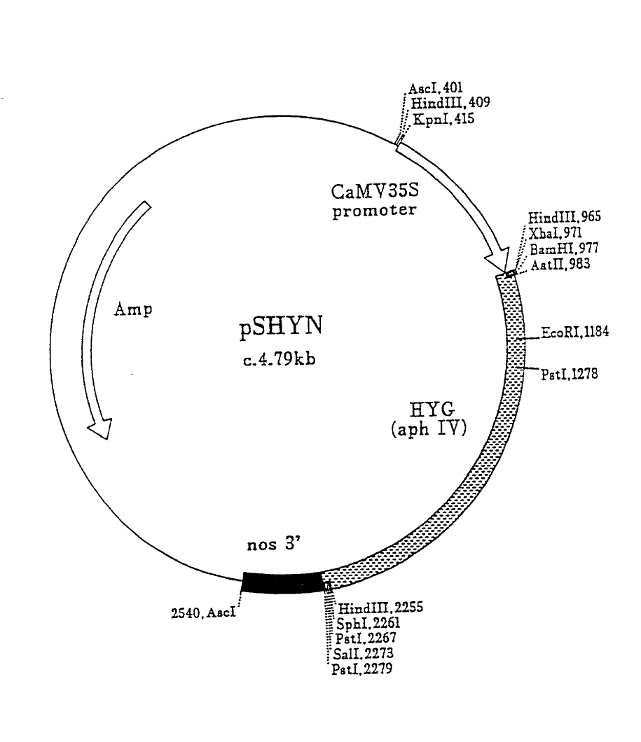

FIG. 2. Plant transformation vector pSHYN, containing hygromycin resistance gene for selection of transformed plants.

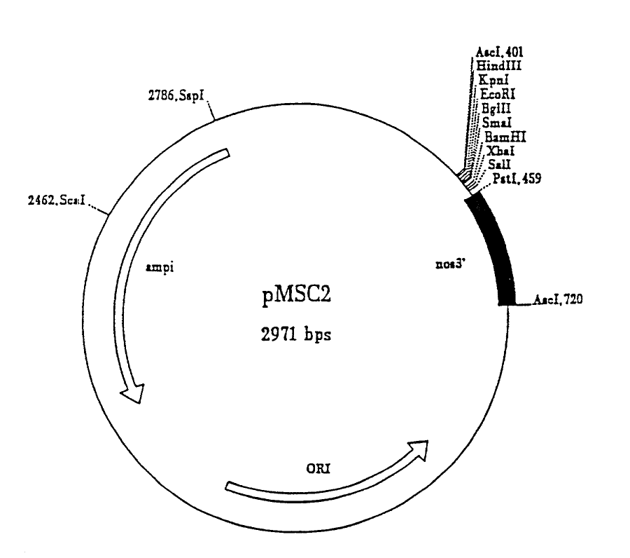

FIG. 3. Plant transformation vector pAN, containing the banana ACC oxidase promoter.

### EXAMPLE 1

Construction of a cDNA Library of Ripening Genes

1.1 Isolation of Messenger RNA

Total RNA was isolated from ripening (24 hours after ethylene treatment) banana pulp (Musa acuminata cv. Grand Nain) as described by Chang et al. Plant Molecular Biology Reporter, Vol. 11(2) 113-116 (1993). Messenger RNA was isolated from total RNA by Oligo(dT)-cellulose chromatography according to Bantle et al., Analytical Biochemistry 72, 413-427 (1976).

1.2 Synthesis of cDNA and Cloning into Vector

The first and second strands of the cDNAs were synthesised from the messenger RNAs using a commercial cDNA synthesis kit (Catalog No. 200450, ZAP Express™ Gold Cloning kit, Stratagene Ltd, Cambridge, Cambs, UK). Double stranded cDNAs were cloned into the ZAP Express™ vector, packaged, mixed with plating bacteria to determine titre and for library screening, following instructions of the suppliers protocol.

1.3 Screening of the cDNA Library from Banana Pulp

The unamplified cDNA library from ripening banana pulp was differentially screened using cDNA from unripe and ripening banana peel tissue. A proportion of the library was plated individually at low density and duplicate plaque lifts made onto Hybond N nylon filters (Amersham) according to the manufacturer's instructions. One filter was hybridised to dCTP radiolabelled cDNA from green fruit and the duplicate filter hybridised to dCTP radiolabelled cDNA from ripening fruit. Hybridization's were at high stringency. Plaques hybridising preferentially with ripening or green radiolabelled cDNA were picked and re-plated for a second round of selection by differential screening. These clones were numbered as ripening up- or down-regulated peel clones. The clones were in-vivo excised from the ZAP express™ vector into the pBK-CMV phagemid vector using the ExAssist™ interference-resistant helper phage, following instructions from manufacturers protocol.

1.4 Characterisation of the Ripening Pulp cDNA Library and the Ripening-related Clones

The ripening cDNA library from pulp tissue were prepared with an efficiency of 3.2×10⁵ plaque-forming units per microgram of cDNA. The sizes of the inserts in the peel library was 0.4-6.7 Kb with a mean size insert of 1.47 Kb.

From the 250 plaques used in the first screen, 73 putative ripening-related clones were obtained. These 73 clones were partially sequenced using the ABI PRISMTM Dye Terminator Cycle Sequencing Ready Reaction kit with AmpliTaq® DNA polymerise (Applied Biosystems, Warrington, Cheshire, UK) with forward primers specific for the pBK-CMV vector. From these, the following ripening-related clones were selected. Comparisons of these sequences in the EMBL database using GCG (‘Wisconsing’) software has identified homologies for the clones listed in TABLE 1 below.

### EXAMPLE 2

Construction of Truncated Sense RNA Vectors With the Maize Polyubiquitin Promoter

A vector is constructed using the sequences corresponding to a fragment of the inserts of one of the sequences 1 to 57. This fragment is synthesised by polymerase chain reaction using synthetic primers incorporating BamHI restriction sites suitable for cloning between a maize UBI polyubiquitin promoter (Christensen et al, 1992, Plant Molecular Biology, 18:675-689) and a nopaline synthase 3′end termination sequences in the vector pUN (FIG. 1.).

The truncated sense expression cassette is excised by digestion with AscI, the ends of the fragment are made flush with T4 polymerase and it is cloned into the vector pSHYN (FIG. 2.) which has been cut with KpnI and the ends made flush with Klenow polymerase. pSHYN contains hnygromycin resistance gene for selection of transformed plants.

After synthesis of the vector, the structure and orientation of the sequences are confirmed by DNA sequence analysis.

### EXAMPLE 3

Construction of Truncated Sense RNA Vectors With a Fruit Enhanced Promoter

The 1386 bp HindIII fragment containing the banana ACC oxidase promoter (UK. Application No. 9607700.3) is cloned the HindIII site in pMSC2 (FIG. 3.) to give the vector pAN.

A vector is constructed using the sequences corresponding to a fragment of the inserts of one of the sequences 1 to 57. This fragment is synthesised by polymerase chain reaction using synthetic primers incorporating BamHI restriction sites suitable for cloning between a maize UBI polyubiquitin promoter (Christensen et al, 1992, Plant Molecular Biology, 18:675-689) and a nopaline synthase 3′end termination sequences in the vector pAN. The truncated sense expression cassette is excised by digestion with AscI, the ends of the fragment are made flush with T4 polymerase and it is cloned into the vector pSHYN (FIG. 2.) which has been cut with KpnI and the ends made flush with Klenow polymerase. pSHYN contains hygromycin resistance gene for selection of transformed plants.

After synthesis of the vector, the structure and orientation of the sequences are confirmed by DNA sequence analysis.

### EXAMPLE 4

Construction of an Over-expression Vector With the Maize Polyubiquitin Promoter

The complete sequence of a ripening-related cDNA containing a full open-reading frame is inserted into the vectors described in EXAMPLE 2.

### EXAMPLE 5

Construction of an Over-expression Vector With a Fruit Enhanced Promoter

The complete sequence of a ripening-related cDNA containing a full open-reading frame is inserted into the vectors described in EXAMPLE 3.

### EXAMPLE 6

Generation of Transformed Musa Plants

Transformed Musa plants containing the vectors are produced by the method described in Sagi et al. (1995) Biotechnology. Vol. 13 pp 481-485. Regenerated transformed plants are identified by their ability to grow on hygromycin and grown to maturity. Ripening fruit are analysed for a modulation in their ripening related or senescence characteristics.

Other suitable transformation methods for banana are described in Sagi et al. (1994) Plant Cell Reports. Vol. 13. pp 262-266. and May et al. (1995) Biotechnology. Vol. 13 pp 486-492.

TABLE 1

| Sequence | | Clone | Size | Published Sequence | % | Sequence | |
|---|---|---|---|---|---|---|---|
| Identity | Group | no. | Kb | gene Identity | Identity | Bp | Published Sequences |
| SEQ-ID-NO-1 | Pulp | U-U9 | 1 | Isoflavone Reductase | 61.6 | 714 | Potato (Solanum |
| | Upregulated | | | | | | tuberosum), X92075 |
| SEQ-ID-NO-2 | Pulp | U-U17 | 1 | Isoflavone Reductase | 62.9 | 770 | Potato (Solanum |
| | Upregulated | | | | | | tuberosum), X92075 |
| SEQ-ID-NO-3 | Pulp | U-U66 | 1.1 | Isoflavonoid | 60.0 | 722 | Arabidopsis thailiana, |
| | Upregulated | | | Reductase | | | Z49777 |
| SEQ-ID-NO-4 | Pulp | U-U104 | 1 | Isoflavonoid | 60.6 | 696 | Arabidopsis thailiana |
| | Upregulated | | | Reductase | | | Z49777 |
| SEQ-ID-NO-5 | Pulp | U-U13 | 1.1 | Beta-1,3-Glucanase | 58.5 | 585 | Soybean (Glycine max), |
| | Upregulated | | | | | | A26451 |
| SEQ-ID-NO-6 | Pulp | U-U136 | 1.3 | Beta-1,3-Glucanase | 59.8 | 800 | Barley (Hordeum vulgare), |
| | Upregulated | | | | | | M91814 |
| SEQ-ID-NO-7 | Pulp | U-U21 | 1.8 | Transcriptional | 54.3 | 311 | Zea mays, L13454 |
| | Upregulated | | | Activator | | | |
| SEQ-ID-NO-8 | Pulp | U-U31 | 0.55 | 5- | 67.0 | 467 | Orchid (Phalaenopsis sp.), |
| | Upregulated | | | adenosylhomocysteine | | | X79905 |
| | | | | Hydrolase | | | |
| SEQ-ID-NO-9 | Pulp | U-U131 | 1.8 | 5-adenosyl L- | 79.8 | 699 | Wheat (Triticum aestivum), |
| | Upregulated | | | homocysteine | | | L11872 |
| | | | | Hydrolase | | | |
| SEQ-ID-NO-10 | Pulp | U-U32 | 0.8 | Beta-amylase | 54.7 | 854 | Zea mays, Z25871 |
| | Upregulated | | | | | | |
| SEQ-ID-NO-11 | Pulp | U-U55 | 0.8 | O-methyl transferases | 60.1 | 223 | Zea mays, L14063 |
| | Upregulated | | | | | | |
| SEQ-ID-NO-12 | Pulp | U-U72 | 0.8 | P-methyl transferase | 60.6 | 226 | Zea mays, L14063 |
| | Upregulated | | | | | | |
| SEQ-ID-NO-13 | Pulp | U-U68 | 1.8 | Pectate Lyase | 57.9 | 394 | Lilium longiflorum, Z17328 |
| | Upregulated | | | | | | |
| SEQ-ID-NO-14 | Pulp | U-U69 | 1.7 | Pectate Lyase | 66.1 | 516 | Zea mays, L20140 |
| | Upregulated | | | | | | |
| SEQ-ID-NO-15 | Pulp | U-U84 | 1.1 | Pectate Lyase | 65.8 | 736 | Lycopersicon esculentum, |
| | Upregulated | | | | | | X55193 |
| SEQ-ID-NO-16 | Pulp | U-U89 | 1.6 | Pectate Lyase | 54.8 | 354 | Nicotiana tabacum, X67158 |
| | Upregulated | | | | | | |
| SEQ-ID-NO-17 | Pulp | U115 | 1.5 | Pectate Lyase | 67.3 | 508 | Zea mays, L20140 |
| | Upregulated | | | | | | |
| SEQ-ID-NO-18 | Pulp | U-U117 | 1.7 | Pectate Lyase | 55.0 | 333 | Lilium longiflorum, Z17328 |
| | Upregulated | | | | | | |
| SEQ-ID-NO-19 | Pulp | U-U80 | 0.6 | Vicilin-like storage | 53.7 | 387 | Arabidopsis thailiana, |
| | Upregulated | | | protein | | | T13642 |
| SEQ-ID-NO-20 | Pulp | U-U90 | 1.8 | Glutamate | 72.5 | 735 | Petunia hybrida, L16797 |
| | Upregulated | | | Descarboxylase | | | |
| SEQ-ID-NO-21 | Pulp | U-U92 | 1.8 | Glutamate | 71.8 | 740 | Arabidopsis thailiana, |
| | Upregulated | | | Descarboxylase | | | U10034 |
| SEQ-ID-NO-22 | Pulp | U-U91 | 1.1 | Aconitase | 76.4 | 766 | Pumpkin (Cucurbita sp.), |
| | Upregulated | | | | | | D29629 |
| SEQ-ID-NO-23 | Pulp | U-U96 | 0.9 | Express Sequence | 69.0 | 452 | Arabidopsis thailiana, |
| | Upregulated | | | Tag. | | | H36910 |
| SEQ-ID-NO-24 | Pup | U-U103 | 2 | Cell Wall Invertase | 66.0 | 567 | Zea mays, U17695 |
| | Upregulated | | | | | | |
| SEQ-ID-NO-25 | Pulp | U-U93 | 3.5 | Heat Shock Protein | 76.4 | 711 | Lycopersicon esculentum, |
| | Upregulated | | | cognate | | | X54030 |
| SEQ-ID-NO-26 | Pulp | U-U125 | 1.8 | Heat Shock Protein | 71.0 | 662 | Spinacia oleracea, L26243 |
| | Upregulated | | | | | | |
| SEQ ID-NO-27 | Pulp | U-U105 | 0.9 | Hexaubiquitin Protein | 64.1 | 237 | Sunflower (Helianthus |
| | Upregulated | | | | | | annuus), X57004 |
| SEQ-ID-NO-28 | Pulp | U-U116 | 0.8 | Polyubiquitin | 81.0 | 625 | Rice (Oryza sativa), X76064 |
| | Upregulated | | | | | | |
| SEQ-ID-NO-29 | Pulp | U-U120 | 0.9 | Extensin | 56.9 | 576 | Almond (Prunus |
| | Upregulated | | | | | | amygdalus), X65718 |
| SEQ-ID-NO-30 | Pulp | U-U126 | 1.2 | Basic Chitinase | 63.2 | 506 | Cowpea (Vigna |
| | Upregulated | | | | | | unguiculata), X88801 |
| SEQ-ID-NO-31 | Pulp | U-U129 | 1.3 | Beta-glucosidase | 60.9 | 517 | White clover (Trifolium |
| | Upregulated | | | | | | repens), X56733 |
| SEQ-ID-NO-32 | Pulp | U-U130 | 1.3 | Chlorophyll a/b | 52.0 | 415 | Rice (Oryza sativa), D00641 |
| | Upregulated | | | binding protein | | | |
| SEQ-ID-NO-33 | Pulp | U-U43 | 2.5 | Fruit sp pp involved in | 53.8 | 396 | Tomato (Lycopersicon |
| | Upregulated | | | maturation | | | esculentum), X13743 |
| SEQ-ID-NO-34 | Pulp | U-U70 | 0.6 | Root specific protein | 63.6 | 420 | Rice (Oryza sativa), L27208 |
| | Upregulated | | | | | | |
| SEQ-ID-NO-35 | Pulp | U-U16 | 0.6 | — | — | — | No published sequence |
| | Upregulated | | | | | | similarity |
| SEQ-ID-NO-36 | Pulp | U-U30 | 0.8 | — | — | — | — |
| | Upregulated | | | | | | |
| SEQ-ID-NO-37 | Pulp | U-U40 | 10 | — | — | — | — |
| | Upregulated | | | | | | |
| SEQ-ID-NO-38 | Pulp | U-U108 | 1.1 | — | — | — | — |
| | Upregulated | | | | | | |
| SEQ-ID-NO-39 | Pulp | U-U128 | 2.2 | — | — | — | — |
| | Upregulated | | | | | | |
| SEQ-ID-NO-40 | Pulp | U-D4 | 1.3 | Granule Bond Starch | 62.3 | 621 | Cassava (Manihot |
| | Down | | | Synthase | | | esculenta), X74160 |
| | regulated | | | | | | |
| SEQ-ID-NO-41 | Pulp | U-D10 | 1.3 | Starch Synthase | 68.8 | 506 | Pea (Pisum sativum), |
| | Down | | | | | | X88789 |
| | regulated | | | | | | |
| SEQ-ID-NO-42 | Pulp | U-D13 | 1.3 | Starch Synthase | 67.4 | 454 | Pea (Pisum sativum), |
| | Down | | | | | | X88789 |
| | regulated | | | | | | |
| SEQ-ID-NO-43 | Pulp | U-D66 | 1.3 | Granule Bond Starch | 64.1 | 669 | Cassava (Manihot |
| | Down | | | Synthase | | | esculenta), X74160 |
| | regulated | | | | | | |
| SEQ-ID-NO-44 | Pulp | U-D111 | 1.3 | Starch Synthase | 66.4 | 655 | Cassava (Manihot |
| | Down | | | | | | esculenta), X74160 |
| | regulated | | | | | | |
| SEQ-ID-NO-45 | Pulp | U-D112 | 2.3 | Granule Bond Starch | 68.4 | 196 | Potato (Solanum |
| | Down | | | Synthase | | | tuberosum), X58453 |
| | regulated | | | | | | |
| SEQ-ID-NO-46 | Pulp | U-D39 | 1.1 | Antifungal Protein | 69.1 | 517 | Zea mays, U06831 |
| | regulated | | | | | | |
| SEQ-ID-NO-47 | Pulp | U-D50 | 1.3 | Basic Chitinase | 60.2 | 678 | Cowpea (Vigna |
| | Down | | | | | | unguiculata), X88801 |
| | regulated | | | | | | |
| SEQ-ID-NO-48 | Pulp | U-D86 | 1.7 | Basic Chitinase | 63 | 578 | Cowpea (Vigna |
| | Down | | | | | | unguiculata), X88801 |
| | regulated | | | | | | |
| SEQ-ID-NO-49 | Pulp | U-D90 | 1.3 | Basic Chitinase | 61.8 | 670 | Cowpea (Vigna |
| | Down | | | | | | unguiculata), X88801 |
| | regulated | | | | | | |
| SEQ-ID-NO-50 | Pulp | U-D93 | 0.7 | Basic Chitinase | 61.6 | 701 | Cowpea (Vigna |
| | Down | | | | | | unguiculata), X88801 |
| | regulated | | | | | | |
| SEQ-ID-NO-51 | Pulp | U-D61 | 1 | Beta-glucanase | 61.9 | 795 | Barley (Hordeum vulgare), |
| | Down | | | | | | X52572 |
| | regulated | | | | | | |
| SEQ-ID-NO-52 | Pulp | U-D63 | 2.3 | Nodulin Gene | 50.4 | 637 | Lucerne (Medicago sativa), |
| | Down | | | | | | X13287 |
| | regulated | | | | | | |
| SEQ-ID-NO-53 | Pulp | U-D65 | 0.9 | Extensin | 56.7 | 556 | Cowpea (Vigna |
| | Down | | | | | | unguiculata), X91836 |
| | regulated | | | | | | |
| SEQ-ID-NO-54 | Pulp | U-D84 | 0.8 | Extensin | 58.3 | 492 | Cowpea (Vigna |
| | Down | | | | | | unguiculata), X91836 |
| | regulated | | | | | | |
| SEQ-ID-NO-55 | Pulp | U-D75 | 1.2 | Wali 7 | 74.4 | 703 | Wheat (Triticum aestivum), |
| | Down | | | | | | L28008 |
| | regulated | | | | | | |
| SEQ-ID-NO-56 | Pulp | U-D83 | 1.2 | Wali 7 | 72.8 | 743 | Wheat (Triticum aestivum), |
| | Down | | | | | | L28008 |
| | regulated | | | | | | |
| SEQ-ID-NO-57 | Pulp | U-D64 | 2.3 | — | — | — | — |
| | Down | | | | | | |
| | regulated | | | | | | |

1. A method of modulating the ripening or tissue senescence process in plants of the genus Musa comprising inserting into plant material at least one polynucleotide sequence selected from the sequences depicted as SEQ ID Nos 1-57, regenerating said plant material and selecting from the transformed regenerants, plants with modulated ripening or tissue senescence characteristics.

2. A method according to claim 1 wherein the polynucleotide is obtained from the cDNA library having the NCIMB Accession Number 40814.

3. A method of modulating the ripening or tissue senescence process in plants of the genus Musa comprising inserting into plant material at least one polynucleotide sequence or a fragment thereof, obtainable by hybridisation, from the cDNA library having the NCIMB Accession Number 40814, by the use of at least one of the sequences depicted as SEQ ID Nos 1-57 as oligonucleotide probes, said hybridisation being conducted at a temperature from 60° C. to 65° C. in 0.3 strength citrate buffered saline containing 0.1% SDS followed by rinsing at the same temperature with 0.3 strength citrate buffered saline containing 0.1% SDS, regenerating said plant material and selecting from the transformed regenerants, plants with modulated ripening or tissue senescence characteristics.

4. A method according to claim 1 or 2 or 3, characterised in that the said polynucleotide modulates the production of pectate lyase.

5. A method according to claim 4 in which the polynucleotide sequence comprises at least one of the sequences depicted in the sequence listings as SEQ ID Nos. 13-18.

6. A method according to any preceding claim wherein the plant material is transformed using the Agrobacterium, microparticle bombardment, fibre mediated or direct insertion method.

7. Plants, their progeny and seed and material obtained from said plants, produced according to a method as claimed in claims 1 to 6.

8. A vector functional in plants comprising a promoter region which is operable in plant cells, a polynucleotide sequence defined in claims 1 to 3 and a transcription termination sequence.

9. A vector according to claim 8 wherein the promoter is constitutive, developmentally regulated or switchable.

10. A vector according to claim 9 wherein the promoter is tissue specific or organ specific.

11. A genetically modified banana produced via the method according to claims 1 to 6 having altered fruit characteristics when compared with a banana which is not transformed with at least one of the polynucleotide sequences described in claims 1 to 3.

12. A method of controlling plant pathogens comprising the application of an anti-pathogenic agent to plants, characterised in that plants to which the said agent is applied are plants according to claim 7.

13. A polynucleotide associated with fruit ripening selected from SEQ ID Nos. 1-57.

14. A fruit of a plant of the genus Musa having a retarded ripening phenotype generated by inserting into the genome of the said plant at least one of the polynucleotide(s) as claimed in claim 13.

15. A method, polynucleotide, plant, its progeny, seed and material obtained from said plants and a banana substantially as hereinbefore described with reference to the accompanying drawings and figures.
